(19) United States
(12) Patent Application Publication
(10) Pub. No.: US 2007/0218525 A1
(43) Pub. Date: Sep. 20, 2007

(54) Process for Producing Dipeptides

(75) Inventors: Kuniki Kino, Chiba (JP); Masaru Sato, Kanagawa (JP)

Correspondence Address: NIXON & VANDERHYE, PC, 901 NORTH GLEBE ROAD, 11TH FLOOR, ARLINGTON, VA, 22203, US

(73) Assignee: Kyowa Hakko Kogyo Co., Ltd.

(21) Appl. No.: 11/547,491

(22) PCT Filed: Mar. 4, 2005
(86) PCT No.: PCT/JP05/03718

§ 371 Date: Sep. 29, 2006

(30) Foreign Application Priority Data
Mar. 29, 2004 (JP) 2004-093953

(51) Int. Cl. C12P 1/04

(52) U.S. Cl. 435/69.1; 435/71.1

(57) ABSTRACT

The present invention provides a process for producing a dipeptide other than D-alanyl-D-alanine and D-alanyl-D-serine using, as an enzyme source, a culture of a microorganism having the ability to produce a protein having D-alanine-D-alanine ligase activity or a treated matter of the culture, and a process for producing a dipeptide comprising D-amino acid using, as enzyme sources, a culture of a microorganism having the ability to produce the D-amino acid or a treated matter of the culture and a culture of a microorganism having the ability to produce a protein having D-alanine-D-alanine ligase activity or a treated matter of the culture.

### TECHNICAL FIELD

The present invention relates to an enzymatic process for producing a dipeptide.

### BACKGROUND ART

Known methods for producing dipeptides comprising D-amino acid include chemical synthesis and enzymatic methods. In the synthesis of dipeptides comprising D-amino acid by the chemical synthesis methods, operations such as introduction and removal of protective groups for functional groups are necessary, and racemates are also formed. The chemical synthesis methods are thus considered to be disadvantageous in respect of cost and efficiency. They are unfavorable also from the viewpoint of environmental hygiene because of the use of large amounts of organic solvents and the like.

As to the synthesis of dipeptides comprising D-amino acid by the enzymatic methods, the following methods are known: a method utilizing reverse reaction of peptidase (see non-patent publication No. 1); methods utilizing aminoacyl transferase (see non-patent publication Nos. 2 and 3); and a method utilizing α-amino acid esterase (see non-patent publication No. 4).

However, the method utilizing reverse reaction of peptidase requires introduction and removal of protective groups for functional groups of amino acids used as substrates, which causes difficulties in raising the efficiency of reaction to form dipeptides comprising D-amino acid and in preventing dipeptide hydrolysis reaction. The methods utilizing aminoacyl transferase are not efficient, for they require the process of esterification of amino acids used as substrates. The method utilizing α-amino acid esterase has the defect that it also requires the process of esterification of amino acids used as substrates, which causes difficulties in raising the efficiency of reaction to form dipeptides comprising D-amino acid and in preventing peptide hydrolysis reaction.

D-alanine-D-alanine ligase is known to have the activity to form D-alanyl-D-alanine and D-alanyl-D-serine using D-alanine, and D-alanine and D-serine, as respective substrates (see non-patent publication No. 5). However, it is not known that the enzyme has the activity to form dipeptides comprising D-amino acid other than D-alanyl-D-alanine and D-alanyl-D-serine, and a process for producing dipeptides comprising D-amino acid other than D-alanyl-D-alanine and D-alanyl-D-serine using, as an enzyme source, a culture of a microorganism having the ability to produce the enzyme is not known either.

Known examples of proteins having the activity to form D-amino acid include hydantoinase having the activity to form D-amino acid from 5-substituted-DL-hydantoin (see non-patent publication No. 6), D-aminoacylase having the activity to form D-amino acid from N-acyl-DL-amino acid (see non-patent publication No. 7), D-amino-acid amidase having the activity to form D-amino acid from DL-amino acid amide (see non-patent publication No. 8), D-amino-acid aminotransferase having the activity to form D-amino acid from α-keto acid (see non-patent publication No. 9) and amino-acid racemase having the activity to form D-amino acid from L-α-amino acid. Low-substrate-specific amino-acid racemase, which is classified as EC 5.1.1.10 and whose substrate specificity is very low, is an enzyme useful for the industrial production of racemates of various amino acids. The microorganisms so far reported to produce this enzyme are microorganisms belonging to the genera Pseudomonas (see non-patent publication Nos. 10 and 11) and Aeromonas (see non-patent publication No. 12). However, their activity is often weak, and it is difficult to fully compensate this defect by improvement of culturing method or acquisition of a mutant strain.

With regard to the proteins having low-substrate-specific amino-acid racemase activity, there is a report on the amino acid sequence of the active site of the protein derived from Pseudomonas putida IFO12996 (see non-patent publication No. 13). There is also a report that DNA encoding the enzyme was obtained and introduced into a microorganism such as Escherichia coli to express a protein having the enzyme activity (see patent publication No. 1).

However, it is not known that dipeptides comprising D-amino acid are formed from amino acids including L-α-amino acid by using a protein having amino-acid racemase activity and a protein having D-alanine-D-alanine ligase activity in combination.

Non-patent publication No. 1:

J. Mol. Cat. B Enzymatic, 6, 379-386 (1999)
   Non-patent publication No. 2:
J. Biochem., 131, 247-254 (2002)
   Non-patent publication No. 3:
J. Biosci. Bioeng., 89, 195-306 (2000)
   Non-patent publication No. 4:
J. Biochem., 130, 119-126 (2001)
   Non-patent publication No. 5:
Chem. Biol., 5, 197-207 (1998)
   Non-patent publication No. 6:
Biotechnol. Bioeng., 23, 2173-2183 (1981)
   Non-patent publication No. 7:
Agric. Biol. Chem., 44, 1089-1095 (1980)
   Non-patent publication No. 8:
Biochem. Biophys. Res. Commun., 162, 470-474 (1989)
   Non-patent publication No. 9:
FEBS Lett., 55, 265-267 (1975)
   Non-patent publication No. 10:
Methods Enzymol., 17B, 629-636 (1971)
   Non-patent publication No. 11:
J. Bacteriol., 175, 4213-4217 (1993)
   Non-patent publication No. 12:
Agric. Biol. Chem., 51, 173-180 (1987)
   Non-patent publication No. 13:
Biochemistry, 23, 5195-5201 (1984)
   Patent publication No. 1:
WO03/074690 pamphlet

### DISCLOSURE OF THE INVENTION

### Problems to be Solved by the Invention

An object of the present invention is to provide a process for producing a dipeptide other than D-alanyl-D-alanine and D-alanyl-D-serine using, as an enzyme source, a culture of a microorganism having the ability to produce a protein having D-alanine-D-alanine ligase activity or a treated matter of the culture, and a process for producing a dipeptide comprising D-amino acid using, as enzyme sources, a culture of a microorganism having the ability to produce the D-amino acid or a treated matter of the culture, and a culture of a microorganism having the ability to produce a protein having D-alanine-D-alanine ligase activity or a treated matter of the culture.

### Means for Solving the Problems

The present invention relates to the following (1) to (18).
(1) A process for producing a dipeptide, which comprises: allowing an enzyme source, ATP and one or more kinds of amino acids selected from the group consisting of D-amino acids and glycine (provided that said one or more kinds of amino acids are not D-alanine alone or a combination of D-alanine and D-serine) to be present in an aqueous medium, said enzyme source being a culture of a microorganism having the ability to produce a protein having D-alanine-D-alanine ligase activity or a treated matter of the culture; allowing the dipeptide to form and accumulate in the aqueous medium; and recovering the dipeptide from the aqueous medium.
(2) The process according to the above (1), wherein the D-amino acid is produced by using a culture of a microorganism having the ability to produce the D-amino acid or a treated matter of the culture as an enzyme source.
(3) A process for producing a dipeptide comprising D-amino acid which comprises: allowing enzyme sources, ATP and a substance which is converted into the D-amino acid by a culture of a microorganism having the ability to produce the D-amino acid or a treated matter of the culture to be present in an aqueous medium, said enzyme sources being a culture of a microorganism having the ability to produce the D-amino acid or a treated matter of the culture, and a culture of a microorganism having the ability to produce a protein having D-alanine-D-alanine ligase activity or a treated matter of the culture; allowing the dipeptide comprising D-amino acid to form and accumulate in the aqueous medium; and recovering the dipeptide from the aqueous medium.
(4) The process according to any one of the above (1) to (3), wherein the protein having D-alanine-D-alanine ligase activity is a protein selected from the group consisting of the following. [1] to [3]:
   [1] a protein having the amino acid sequence shown in any of SEQ ID NOS: 1 to 5;
   [2] a protein consisting of an amino acid sequence wherein one or more amino acid residues are deleted, substituted or added in the amino acid sequence shown in any of SEQ ID NOS: 1 to 5 and having D-alanine-D-alanine ligase activity; and
   [3] a protein consisting of an amino acid sequence which has 80% or more homology to the amino acid sequence shown in any of SEQ ID NOS: 1 to 5 and having D-alanine-D-alanine ligase activity.
(5) The process according to any one of the above (1) to (3), wherein the microorganism having the ability to produce a protein having D-alanine-D-alanine ligase activity is a microorganism obtainable by introducing DNA encoding the protein having D-alanine-D-alanine ligase activity into a host cell.
(6) The process according to the above (5), wherein the DNA encoding a protein having .D-alanine-D-alanine ligase activity is DNA selected from the group consisting of the following [1] to [4]:
   [1] DNA encoding a protein having the amino acid sequence shown in any of SEQ ID NOS: 1 to 5;
   [2] DNA having the nucleotide sequence shown in any of SEQ ID NOS: 6 to 10;
   [3] DNA which hybridizes with DNA having a nucleotide sequence complementary to the nucleotide sequence shown in any of SEQ ID NOS: 6 to 10 under stringent conditions and which encodes a protein having D-alanine-D-alanine ligase activity; and
   [4] DNA consisting of a nucleotide sequence which has 90% or more homology to the nucleotide sequence shown in any of SEQ ID NOS: 6 to 10 and encoding a protein having D-alanine-D-alanine ligase activity.
(7) The process according to the above (2) or (3), wherein the microorganism having the ability to produce D-amino acid is a microorganism having the ability to produce a protein having the activity of hydantoinase, D-aminoacylase, D-amino-acid amidase, D-amino-acid transaminase or amino-acid racemase.
(8) The process according to the above (7), wherein the protein having amino-acid racemase activity is a protein having low-substrate-specific amino-acid racemase activity.
(9) The process according to the above (8), wherein the protein having low-substrate-specific amino-acid racemase activity is a protein selected from the group consisting of the following [1] to [3]:
   [1] a protein having the amino acid sequence shown in SEQ ID NO: 11 or 12;
   [2] a protein consisting of an amino acid sequence wherein one or more amino acid residues are deleted, substituted or added in the amino acid sequence shown in SEQ ID NO: 11 or 12 and having low-substrate-specific amino-acid racemase activity; and
   [3] a protein consisting of an amino acid sequence which has 80% or more homology to the amino acid sequence shown in SEQ ID NO: 11 or 12 and having low-substrate-specific amino-acid racemase activity.
(10) The process according to the above (2) and (3), wherein the microorganism having the ability to produce D-amino acid is a microorganism obtainable by introducing DNA encoding a protein having the activity of hydantoinase, D-aminoacylase, D-amino-acid amidase, D-amino-acid aminotransferase or amino-acid racemase into a host cell.
(11) The process according to the above (10), wherein the DNA encoding a protein having amino-acid racemase activity is DNA encoding low-substrate-specific amino-acid racemase.
(12) The process according to the above (11), wherein the DNA encoding a protein having low-substrate-specific amino-acid racemase activity is DNA selected from the group consisting of the following [1] to [4]:
   [1] DNA encoding a protein having the amino acid sequence shown in SEQ ID NO: 11 or 12;
   [2] DNA having the nucleotide sequence shown in SEQ ID NO: 13 or 14;
   [3] DNA which hybridizes with DNA having a nucleotide sequence complementary to the nucleotide sequence shown in SEQ ID NO: 13 or 14 under stringent conditions and which encodes a protein having low-substrate-specific amino-acid racemase activity; and
   [4] DNA consisting of a nucleotide sequence which has 90%, or more homology to the nucleotide sequence shown in SEQ ID NO: 13 or 14 and encoding a protein having low-substrate-specific amino-acid racemase activity.
(13) The process according to any one of the above (1), (2) and (4) to (6), wherein the one or more kinds of amino acids selected from the group consisting of D-amino acids and glycine are D-amino acids selected from the group consisting of D-alanine, D-glutamine, D-glutamic acid, glycine, D-valine, D-leucine, D-isoleucine, D-proline, D-phenylalanine, D-tryptophan, D-methionine, D-serine, D-threonine, D-cysteine, D-asparagine, D-tyrosine, D-lysine, D-arginine, D-histidine, D-aspartic acid and D-ornithine (provided that said one or more kinds of amino acids are not D-alanine alone or a combination of D-alanine and D-serine).
(14) The process according to any one of the above (3) to (12), wherein the substance which is converted into D-amino acid by a culture of a microorganism having the ability to produce the D-amino acid or a treated matter of the culture is one or more kinds of 5-substituted-DL-hydantoin, one or more kinds of N-acyl-DL-amino acids, one or more kinds of DL-amino acid amides, one or more kinds of α-keto acids and one or more kinds of D-amino acids, or one or more kinds of amino acids comprising one or more kinds of L-α-amino acids.
(15) The process according to the above (14), wherein the one or more kinds of amino acids comprising one or more kinds of L-α-amino acids comprise one or more kinds of amino acids selected from the group consisting of L- or D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid and ornithine, and glycine.
(16) The process according to any one of the above (1), (2) and (4) to (12), wherein the dipeptide is a dipeptide represented by formula (I):
   R¹—R² (I)
   (wherein R¹ and R², which may be the same or different, each represent D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid or ornithine, or glycine; provided that both R¹ and R² cannot represent D-alanine at the same time, and when R¹ is D-alanine, R² does not represent D-serine).
(17) The process according to any one of the above (3) to (12), wherein the dipeptide comprising D-amino acid is a dipeptide represented by formula (II):
   R³—R⁴ (II)
   (wherein R³ and R⁴, which may be the same or different, each represent L- or D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid or ornithine, or glycine; provided that both R³ and R⁴ cannot represent L-form of amino acids or glycine at the same time).
(18) The process according to any one of the above (3) to (12), wherein the dipeptide comprising D-amino acid is a dipeptide represented by formula (III):
   R⁵—R⁶ (III)
   (wherein R⁵ and R⁶, which may be the same or different, each represent D-alanine, D-glutamine, D-glutamic acid, D-valine, D-leucine, D-isoleucine, D-proline, D-phenylalanine, D-tryptophan, D-methionine, D-serine, D-threonine, D-cysteine, D-asparagine, D-tyrosine, D-lysine, D-arginine, D-histidine, D-aspartic acid, D-ornithine or glycine; provided that both R⁵ and R⁶ cannot represent glycine at the same time).

### Effect of the Invention

The present invention provides a process for producing a dipeptide other than D-alanyl-D-alanine and D-alanyl-D-serine using, as an enzyme source, a culture of a microorganism having the ability to produce a protein having D-alanine-D-alanine ligase activity or a treated matter of the culture, and a process for producing a dipeptide comprising D-amino acid using, as enzyme sources, a culture of a microorganism having the ability to produce the D-amino acid or a treated matter of the culture, and a culture of a microorganism having the ability to produce a protein having D-alanine-D-alanine ligase activity or a treated matter of the culture.

### Best Modes for Carrying Out the Invention

The present invention is described in detail below.

1. Microorganisms Having the Ability to Produce a Protein Having D-Alanine-D-Alanine Ligase Activity Used in the Process of the Present Invention

The microorganisms having the ability to produce a protein having D-alanine-D-alanine ligase activity used in the process of the present invention may be any microorganisms that have the ability to produce a protein having D-alanine-D-alanine ligase activity.

Many microorganisms have D-alanine-D-alanine ligase (also referred to as ddl) activity, and examples of the microorganisms are those belonging to the genus Escherichia [described in DNA Data Bank of Japan (DDBJ) as having ddlB gene and ddlA gene], those belonging to the genus Oceanobacillus (described in DDBJ as having ddlA gene), those belonging to the genus Synechocystis (described in DDBJ as having ddlA gene), those-belonging to the genus Bacillus [Nature, 390, 249-256 (1997)], those belonging to the genus Corynebacterium (D-alanine-D-alanine ligase and a gene encoding the enzyme are shown under NCBI ACCESSION NO. AP005282), those belonging to the genus Pseudomonas [Microbiology, 142, 79-86 (1996)], those belonging to the genus Helicobacter [Nature, 388, 539-547 (1997)], those belonging to the genus Mycobacterium [Nature, 393, 537-544 (1998)], those belonging to the genus Arabidopsis (the protein represented by NCBI ACCESSION NO. NM_{—}202530 is described as belonging to the family of D-alanine-D-alanine ligase) and those belonging to the genus Thermotoga (described in DDBJ as having a gene encoding D-alanine-D-alanine ligase). Other microorganisms that produce a protein having D-alanine-D-alanine ligase activity can be obtained by searching various kinds of public databases with the name of the enzyme as a keyword.

Microorganisms preferred for use in the present invention are those belonging to the genera Escherichia, Oceanobacillus, Synechocystis and Thermotoga.

Specifically, the microorganisms having the ability to produce a protein having D-alanine-D-alanine ligase activity include the following [1] to [3]:
[1] a microorganism having the ability to produce a protein having the amino acid sequence shown in any of SEQ ID NOS: 1 to 5;
[2] a microorganism having the ability to produce a protein consisting of an amino acid sequence wherein one or more amino acid residues are deleted, substituted or added in the amino acid sequence shown in any of SEQ ID NOS: 1 to 5 and having low-substrate-specific amino-acid racemase activity; and
[3] a microorganism having the ability to produce a protein having 80% or more homology, preferably 90% or more homology, more preferably 95% or more homology, further preferably 98% or more homology, particularly preferably 99% or more homology to the amino acid sequence shown in any of SEQ ID NOS: 1 to 5 and having D-alanine-D-alanine ligase activity.

Specific examples of the microorganisms are Escherichia coli K-12, which is a microorganism having the ability to produce a protein having the amino acid sequence shown in SEQ ID NO: 1 or 2; Oceanobacillus iheyensis HTE831, which is a microorganism having the ability to produce a protein having the amino acid sequence shown in SEQ ID NO: 3; Synechocystis sp. PCC6803, which is a microorganism having the ability to produce a protein having the amino acid sequence shown in SEQ ID NO: 4; and Thermotoga maritima ATCC 43589, which is a microorganism having the ability to produce a protein having the amino acid sequence shown in SEQ ID NO: 5.

The above microorganisms having the ability to produce a protein consisting of an amino acid sequence wherein one or more amino acid residues are deleted, substituted or added and having D-alanine-D-alanine ligase activity include any microorganisms that have the ability to produce the protein, for example, those obtained by Southern analysis using, as a probe, a part or the whole of DNA having a nucleotide sequence complementary to DNA encoding a protein consisting of the amino acid sequence shown in any of SEQ ID NOS: 1 to 5, more specifically, DNA consisting of the nucleotide sequence shown in any of SEQ ID NOS: 6 to 10. An example of a part of DNA used as the above probe is DNA encoding the active center of a protein consisting of the amino acid sequence shown in any of SEQ ID NOS: 1 to 5.

The above Southern analysis refers to a process of carrying out Southern hybridization, using the above DNA as a probe, of a restriction enzyme digest of chromosomal DNA extracted from the cell of a test microorganism by a known method and checking the presence or absence of the positive signal. Specifically, the process is carried out in the following manner. Hybridization is carried out at 65° C. in the presence of 0.7 to 1.0 mol/l sodium chloride using a filter with the restriction enzyme digest of the chromosomal DNA immobilized thereon, and the filter is washed at 65° C. with a 0.1 to 2-fold conc. SSC solution (1-fold conc. SSC. solution: 150 mmol/l sodium chloride and 15 mmol/l sodium citrate), followed by detection of DNA which hybridizes with the probe DNA even after washing. Hybridization can be carried out according to the methods described in Molecular Cloning, Third Edition; Current Protocols in Molecular Biology; DNA Cloning 1: Core Techniques, A Practical Approach, Second Edition, Oxford University (1995), etc. The microorganisms having hybridizable DNA include, for example, those belonging to the genera Escherichia, Oceanobacillus, Synechocystis and Thermotoga.

The microorganisms having the ability to produce a protein having 80% or more homology to the amino acid sequence shown in any of SEQ ID NOS: 1 to 5 and having D-alanine-D-alanine ligase activity include microorganisms having the ability to produce a protein which consists of an amino acid sequence having at least 80% homology, preferably 90% or more homology, more preferably 95% or more homology, further preferably 98% or more homology, particularly preferably 99% or more homology to the amino acid sequence shown in any of SEQ ID NOS: 1 to 5 as calculated by use of algorithm BLAST by Karlin and Altschul [Pro. Natl. Acad. Sci. USA, 90, 5873 (1993)] and FASTA [Methods Enzymol., 183, 63 (1990)] and which has D-alanine-D-alanine ligase activity. Examples of the microorganisms are those belonging to the genera Escherichia, Oceanobacillus, Synechocystis and Thermotoga.

On the basis of the above BLAST and FASTA, programs such as BLASTN and BLASTX have been developed [J. Mol. Biol., 215, 403 (1990)]. When a nucleotide sequence is analyzed by BLASTN on the basis of BLAST, the parameters, for instance, are as follows: score=100 and wordlength=12. When an amino acid sequence is analyzed by BLASTX on the basis of BLAST, the parameters, for instance, are as follows: score=50 and wordlength=3. The homology among nucleotide sequences and amino acid sequences can be calculated in this way. When BLAST and Gapped BLAST programs are used, default parameters of each program are used. The specific techniques for these analyses are known (http://www.ncbi.nlm.nih.gov.).

The microorganisms having the ability to produce a protein having D-alanine-D-alanine ligase activity used in the process of the present invention also include microorganisms which are obtained by treating the above microorganisms having the ability to produce a protein having D-alanine-D-alanine ligase activity by ordinary mutation-inducing means such as a treatment with a mutagen (e.g., N-methyl-N′-nitro-N-nitrosoguanidine) and mutation treatment by UV irradiation or γ-rays irradiation, and then selecting microorganisms showing higher D-alanine-D-alanine ligase activity compared with their parent strain. D-alanine-D-alanine ligase activity can be measured according to a conventional method.

Further, the microorganisms having the ability to produce a protein having D-alanine-D-alanine ligase activity used in the present invention include transformed microorganisms obtainable by introducing DNA encoding D-alanine-D-alanine ligase into a host cell.

The DNAs encoding a protein having D-alanine-D-alanine ligase activity include genes encoding D-alanine-D-alanine ligase (ddl genes) of the above microorganisms having the ability to produce a protein having D-alanine-D-alanine ligase activity which belong to the genera Escherichia, Oceanobacillus, Synechocystis, Bacillus, Corynebacterium, Pseudomonas, Helicobacter, Mycobacterium, Arabidopsis and Thermotoga. The specific nucleotide sequences of the genes can be obtained from various public databases. DNAs encoding D-alanine-D-alanine ligase preferably used in the present invention include DNAs encoding a protein having D-alanine-D-alanine ligase activity derived from microorganisms belonging to the genus Escherichia, Oceanobacillus, Synechocystis or Thermotoga. Specific examples of the DNAs encoding a protein having D-alanine-D-alanine ligase activity are the following [1] to [4]:
[1] DNA encoding a protein having the amino acid sequence shown in any of SEQ ID NOS: 1 to 5;
[2] DNA having the nucleotide sequence shown in any of SEQ ID NOS: 6 to 10;
[3] DNA which hybridizes with DNA having a nucleotide sequence complementary to the nucleotide sequence shown in any of SEQ ID NOS: 6 to 10 under stringent conditions and which encodes a protein having D-alanine-D-alanine ligase activity; and
[4] DNA consisting of a nucleotide sequence which has 90% or more homology, preferably 95% or more homology, more preferably 98% or more homology, further preferably 99% or more homology to the nucleotide sequence shown in any of SEQ ID NOS: 6 to 10 and encoding a protein having D-alanine-D-alanine ligase activity.

“To hybridize” refers to a step of hybridization of DNA with DNA having a specific -nucleotide sequence or a part of the DNA. Therefore, the nucleotide sequence of the DNA having a specific nucleotide sequence or a part of the DNA may be DNA which is long enough to be useful as a probe for Northern or Southern blot analysis or to be used as an oligonucleotide primer for PCR analysis. Preferably, said DNA comprising at least 10, more preferably at least 15 nucleotides can be used as a probe while said DNA include the DNAs having at least 100 nucleotides, preferably 200 or more nucleotides, more preferably 500 or more nucleotides.

The method for hybridization of DNA is well known and the conditions for hybridization can be determined by a person skilled in the art according to the present specification. The hybridization can be carried out according to the methods described in Molecular Cloning, Second Edition, Third Edition (2001); Methods for General and Molecular Bacteriology, ASM Press (1994); Immunology methods manual, Academic press (Molecular), and many other standard textbooks.

Hybridization under the above stringent conditions is preferably carried out, for example, as follows. A filter with DNA immobilized thereon and a probe DNA are incubated in a solution comprising 50% formamide, 5×SSC (750 mM sodium chloride and 75 mM sodium citrate), 50 mM sodium phosphate (pH 7.6), 5× Denhardt's solution, 10% dextran sulfate and 20 μg/l denatured salmon sperm DNA at 42° C. overnight, and after the incubation, the filter is washed in 0.2×SSC solution (ca. 65° C.). Less stringent conditions can also be employed. Modification of the stringent conditions can be made by adjusting the concentration of formamide (the conditions become less stringent as the concentration of formamide is lowered) and by changing the salt concentrations and the temperature conditions. Hybridization under less stringent conditions is carried out, for example, by incubating a filter with DNA immobilized thereon and a probe DNA in a solution comprising 6×SSCE (20×SSCE: 3 mol/l sodium chloride, 0.2 mol/l sodium dihydrogenphosphate and 0.02 mol/l EDTA, pH 7.4), 0.5% SDS, 30% formamide and 100 μg/l denatured salmon sperm DNA at 37° C. overnight, and washing the filter with 1×SSC solution containing 0.1% SDS (50° C.). Hybridization under still less stringent conditions is carried out by using a solution having a high salt concentration (for example, 5×SSC) under the above less stringent conditions, followed by washing.

Various conditions described above can also be established by adding a blocking reagent used to reduce the background of hybridization or changing the reagent. The addition of the above blocking reagent may be accompanied by changes of conditions for hybridization to make the conditions suitable for the purpose.

The above DNA capable of hybridization under stringent conditions includes DNA having at least 90% homology, preferably 95% or more homology, more preferably 98% or more homology, further preferably 99% or more homology to the nucleotide sequence shown in any of SEQ ID NOS: 6 to 10 as calculated by use of programs such as BLAST and FASTA described above based on the above parameters.

2. Microorganisms Having the Ability to Produce D-Amino Acid Used in the Process of the Present Invention

The microorganisms having the ability to produce D-amino acid used in the process of the present invention may be any microorganisms having such ability. Examples of microorganisms include those having the ability to produce hydantoinase having the activity to form D-amino acid from 5-substituted-DL-hydantoin [Biotechnol. Bioeng., 23, 2173-2183 (1981)], D-aminoacylase having the activity to form D-amino acid from N-acyl-DL-amino acid [Agric. Biol. Chem., 44, 1089-1095 (1980)], D-amino-acid amidase having the activity to form D-amino acid from DL-amino acid amide [Biochem. Biophys. Res. Commun., 162, 470-474 (1989)], D-amino-acid aminotransferase having the activity to form D-amino acid from α-keto acid [FEBS Lett., 55, 265-267 (1975)] or amino-acid racemase having the activity to form D-amino acid from L-α-amino acid. Preferred are microorganisms having the ability to produce amino-acid racemase, and more preferred are microorganisms having the ability to produce low-substrate-specific amino-acid racemase.

Examples of the microorganisms having the ability to produce amino-acid racemase include those belonging to the genera Escherichia [Science, 277, 1453-1474 (1997)], Bacillus [Nature, 390, 249-256 (1997)], Pseudomonas [Nature, 406, 959-964 (2000)] and Oceanobacillus [Nucleic Acids Res., 30, 3927-3935 (2002)], and examples of the microorganisms having the ability to produce a protein having low-substrate-specific amino-acid racemase activity include those belonging to the genus Pseudomonas (WO03/074690). Microorganisms having the ability to produce a protein having low-substrate-specific amino-acid racemase activity are preferably used in the present invention.

Specifically, the microorganisms having the ability to produce a protein having low-substrate-specific amino-acid racemase activity include the following [1] to [3]:
[1] a microorganism having the ability to produce a protein having the amino acid sequence shown in SEQ ID NO: 11 or 12;
[2] a microorganism having the ability to produce a protein consisting of an amino acid sequence wherein one or more amino acid residues are deleted, substituted or added in the amino acid sequence shown in SEQ ID NO: 11 or 12 and having low-substrate-specific amino-acid racemase activity; and
[3] a microorganism having the ability to produce a protein having 80% or more homology, preferably 90% or more homology, more preferably 95% or more homology, further preferably 98% or more homology, particularly preferably 99% or more homology to the amino acid sequence shown in SEQ ID NO: 11 or 12 and having low-substrate-specific amino-acid racemase activity.

Specific examples of the microorganisms are Pseudomonas putida ATCC 47954, which is a microorganism having the ability to produce a protein having the amino acid sequence shown in SEQ ID NO: 11, and Pseudomonas putida IFO 12996, which is a microorganism having the ability to produce a protein having the amino acid sequence shown in SEQ ID NO: 12.

The above microorganisms having the ability to produce a protein consisting of an amino acid sequence wherein one or more amino acid residues are deleted, substituted or added and having low-substrate-specific amino-acid racemase activity include any microorganisms that have the ability to produce the protein, for example, microorganisms obtained by Southern analysis using, as a probe, a part or the whole of DNA having a nucleotide sequence complementary to DNA encoding a protein consisting of the amino acid sequence shown in SEQ ID NO: 11 or 12, more specifically, DNA consisting of the nucleotide sequence shown in SEQ ID NO: 13 or 14. An example of a part of DNA used as the above probe is DNA encoding the active center of a protein consisting of the amino acid sequence shown in SEQ ID NO: 11 or 12.

The above Southern analysis is the same analysis as that of the above 1 and is carried out in the same manner as in the above 1, except that the above DNA is used as a probe.

The microorganisms having DNA capable of hybridization include, for example, those belonging to the genus Pseudomonas.

The microorganisms having the ability to produce a protein having 80% or more homology to the amino acid sequence shown in SEQ ID NO: 11 or 12 and having low-substrate-specific amino-acid racemase activity include microorganisms having the ability to produce a protein which consists of an amino acid sequence having at least 80% homology, preferably 90% or more homology, more preferably 95% or more homology, further preferably 98% or more homology, particularly preferably 99% or more homology to the amino acid sequence shown in SEQ ID NO: 11 or 12 as calculated by use of BLAST and FASTA described above and which has low-substrate-specific amino-acid racemase activity. Examples of the microorganisms are those belonging to the genus Pseudomonas.

The microorganisms having the ability to produce D-amino acid used in the process of the present invention also include microorganisms which are obtained by treating the above microorganisms having the ability to produce a protein having amino-acid racemase activity or the like by ordinary mutation-inducing means (A Short Course in Bacterial Genetics) such as a treatment with a mutagen (e.g., N-methyl-N′-nitro-N-nitrosoguanidine) and mutation treatment by UV irradiation or γ-rays irradiation, and then selecting microorganisms showing higher amino-acid racemase activity or the like compared with their parent strain. The ability to produce D-amino acid, for example, amino-acid racemase activity can be measured according to a conventional method.

Further, the microorganisms having the ability to produce D-amino acid used in the present invention include transformed microorganisms obtainable by introducing DNA encoding hydantoinase, D-aminoacylase, D-amino-acid amidase, D-amino-acid aminotransferase or amino-acid racemase into a host cell.

Known examples of DNAs encoding hydantoinase are DNAs described in Japanese Published Unexamined Patent Application No. 330785/02; known examples of DNAs encoding a protein having D-aminoacylase activity are DNAs described in Biosci. Biotech. Biochem., 59, 2115 (1995) and Japanese Published Unexamined Patent Application No. 275688/01; known examples of DNAs encoding a protein having D-amino-acid amidase activity are DNAs described in Applied & Environmental Microbiology, 60, 888-95 (1994); known examples of DNAs encoding a protein having D-amino-acid aminotransferase activity are DNAs described in J. Bacteriol., 185, 2418-2431 (2003) and Biochim. Biophys. Acta, 1350, 38-40 (1997); and known examples of DNAs encoding a protein having amino-acid racemase activity are DNAs derived from microorganisms belonging to the genera Escherichia [Science, 277, 1453-1474 (1997)], Bacillus [Nature, 390, 249-256 (1997)], Pseudomonas [Nature, 406, 959-964 (2000)] and Oceanobacillus [Nucleic Acids Res., 30, 3927-3935 (2002)]. Known examples of DNAs encoding a protein having low-substrate-specific amino-acid racemase activity are DNAs derived from microorganisms belonging to the genus Pseudomonas (WO03/074690). The nucleotide sequences of DNAs encoding amino-acid racemase can be obtained by searching various kinds of DNA databases using the name of the enzyme as a keyword.

Specific examples of the DNAs encoding a protein having low-substrate-specific amino-acid racemase activity preferably used in the present invention include the following [1] to [4]:
[1] DNA encoding a protein having the amino acid sequence shown in SEQ ID NO: 11 or 12;
[2] DNA having the nucleotide sequence shown in SEQ ID NO: 13 or 14;
[3] DNA which hybridizes with DNA having a nucleotide sequence complementary to the nucleotide sequence shown in SEQ ID NO: 13 or 14 under stringent conditions and which encodes a protein having amino-acid racemase activity; and
[4] DNA consisting of a nucleotide sequence which has 90% or more homology, preferably 95% or more homology, more preferably 98% or more homology, further preferably 99% or more homology to the nucleotide sequence shown in SEQ ID NO: 13 or 14 and encoding a protein having amino-acid racemase activity.

The terms “to hybridize” and “stringent conditions” as used above have the same significances as in the above. 1 and the specific method for acquisition of “DNA hybridizing under stringent conditions” is also the same as in the above 1.

The above DNA capable of hybridization under stringent conditions includes, specifically, DNA having at least 90% homology, preferably 95% or more homology, more preferably 98% or more homology, further preferably 99% or more homology to the nucleotide sequence shown in SEQ ID NO: 13 or 14 as calculated by use of programs such as BLAST and FASTA described above based on the above parameters.

3. Preparation of DNAs Used in the Process of the Present Invention

The DNAs used in the process of the present invention, for example, DNA encoding a protein having amino-acid racemase activity and DNA encoding D-alanine-D-alanine ligase can be prepared in the following manner.

The DNA encoding a protein having amino-acid racemase activity can be obtained by Southern hybridization of a chromosomal DNA library from a microorganism, preferably one belonging to the genus Pseudomonas, using a probe designed based on a nucleotide sequence which is registered as a gene encoding amino-acid racemase in a public DNA database such as GenBank or the nucleotide sequence shown in SEQ ID NO: 13 or 14, or by PCR [PCR Protocols, Academic Press (1990)] using primer DNAs designed based on the above known gene encoding amino-acid racemase or the nucleotide sequence shown in SEQ ID NO: 13 or 14 and using, as a template, the chromosomal DNA of a microorganism, preferably one belonging to the genus Pseudomonas.

The DNA encoding D-alanine-D-alanine ligase can be obtained by Southern hybridization of a chromosomal DNA library from a microorganism, preferably one belonging to the genus Escherichia, Oceanobacillus, Synechocystis or Thermotoga, using a probe designed based on a nucleotide sequence which is registered as a gene encoding D-alanine-D-alanine ligase in a public DNA database such as GenBank or the nucleotide sequence shown in any of SEQ ID NOS: 6 to 10, or by PCR using primer DNAs designed based on the above known gene encoding D-alanine-D-alanine ligase or the nucleotide sequence shown in any of SEQ ID NOS: 6 to 10 and using, as a template, the chromosomal DNA of a microorganism, preferably one belonging to the genus Escherichia, Oceanobacillus, Synechocystis or Thermotoga.

The DNA encoding a protein having amino-acid racemase activity or the DNA encoding a protein having D-alanine-D-alanine ligase activity can also be obtained by conducting a search through various gene sequence databases for a sequence having 60% or more homology, preferably 70% or more homology, more preferably 90% or more homology, further preferably 95% or more homology, particularly preferably 98% or more homology, most preferably 99% or more homology to a known gene encoding amino-acid racemase or the nucleotide sequence shown in SEQ ID NO: 13 or 14, or a known gene encoding D-alanine-D-alanine ligase or the nucleotide sequence of DNA encoding the amino acid sequence shown in any of SEQ ID NOS: 6 to 10, and obtaining the desired DNA, based on the nucleotide sequence obtained by the search, from the chromosomal DNA or cDNA library, etc. of an organism having the nucleotide sequence according to the above-described method.

The obtained DNA, as such or after cleavage with appropriate restriction enzymes, is inserted into a vector by a conventional method, and the obtained recombinant DNA is introduced into a host cell. Then, the nucleotide sequence of the DNA can be determined by a conventional sequencing method such as the dideoxy method [Proc. Natl. Acad. Sci., USA, 74, 5463 (1977)] or by using a nucleotide sequencer such as 373A DNA Sequencer (Perkin-Elmer Corp.).

In cases where the obtained DNA is found to be a partial DNA by the analysis of nucleotide sequence, the full length DNA can be obtained by Southern hybridization of a chromosomal DNA library using the partial DNA as a probe.

It is also possible to prepare the desired DNA by chemical synthesis using a DNA synthesizer (e.g., Model 8905, PerSeptive Biosystems) based on the determined nucleotide sequence of the DNA.

Examples of the DNAs encoding a protein having amino-acid racemase activity that can be obtained by the above-described method are DNAs having the nucleotide sequences shown in SEQ ID NOS: 13 and 14, and examples of the DNAs encoding D-alanine-D-alanine ligase thus obtained are DNAs having the nucleotide sequences shown in SEQ ID NOS: 6 to 10.

4. Preparation of Microorganisms Obtainable by Introducing DNA Used in the Present Invention into a Host Cell

The microorganisms obtainable by introducing the DNA used in the present invention into a host cell, for example, a microorganism obtainable by introducing DNA encoding a protein having amino-acid racemase activity into a host cell and a microorganism obtainable by introducing DNA encoding a protein having D-alanine-D-alanine ligase activity into a host cell can be obtainable by introducing the DNA obtainable in the above 3 into a host cell by a conventional method. The microorganisms may have either the DNA encoding a protein having amino-acid racemase activity or the DNA encoding a protein having D-alanine-D-alanine ligase activity, or both the DNAs at the same time to be used in the process of the present invention.

On the basis of the DNA obtained in the above 3, a DNA fragment of an appropriate length comprising a region encoding a protein is prepared according to need. A microorganism having enhanced productivity of the protein can be prepared by replacing a nucleotide in the nucleotide sequence of the region encoding the protein so as to make a codon most suitable for the expression in a host cell.

The DNA fragment is inserted downstream of a promoter in an appropriate expression vector to prepare a recombinant DNA.

A transformant producing the protein of the present invention can be obtained by introducing the recombinant DNA into a host cell suited for the expression vector.

As the host cell, any microorganisms that are capable of expressing the desired gene can be used.

The expression vectors that can be employed are those capable of autonomous replication or integration into the chromosome in a microorganism and comprising a promoter at a position appropriate for the transcription of the DNA of the present invention or the DNA used in the process of the present invention.

When a procaryote such as a bacterium is used as the host cell, it is preferred that the recombinant DNA comprising the DNA encoding a protein having amino-acid racemase activity or the DNA encoding a protein having D-alanine-D-alanine ligase activity (hereinafter referred to as DNA encoding amino-acid racemase or D-alanine-D-alanine ligase) is a recombinant DNA which is capable of autonomous replication in the procaryote and which comprises a promoter, a ribosome binding sequence, the DNA of the present invention or the DNA used in the process of the present invention, and a transcription termination sequence. The recombinant DNA may further comprise a gene regulating the promoter.

Examples of suitable expression vectors are pBTrp2, pBTac1 and pBTac2 (products of Boehringer Mannheim GmbH), pHelix1 (Roche Diagnostics Corp.), pKK233-2 (Amersham Pharmacia Biotech), pSE280 (Invitrogen Corp.), pGEMEX-1 (Promega Corp.), pQE-8 (Qiagen, Inc.), pET-3 and pET-30Xa/LIC (products of Novagen, Inc.), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Bio Inc.), pUC118 (Takara Bio Inc.), pPA1 (Japanese Published Unexamined Patent Application No. 233798/88) and pJB861 [Plasmid, 38, 35-51 (1997)].

As the promoter, any promoters capable of functioning in host cells such as Escherichia coli can be used. For example, promoters derived from Escherichia coli or phage, such as trp promoter (Pₜᵣₚ), lac promoter (P_{lac}), P_{L} promoter, P_{R} promoter and P_{SE} promoter, SPO1 promoter, SPO2 promoter, penP promoter and Pm promoter can be used. Artificially designed and modified promoters such as a promoter in which two Pₜᵣₚs are combined in tandem, tac promoter, lacT7 promoter and letI promoter, etc. can also be used.

Also useful is P54-6 promoter for the expression in microorganisms belonging to the genus Corynebacterium [Appl. Microbiol. Biotechnol., 53, 674-679 (2000)].

It is preferred to use a plasmid in which the distance between the Shine-Dalgarno sequence (ribosome binding sequence) and the initiation codon is adjusted to an appropriate length (e.g., 6 to 18 nucleotides).

In the recombinant DNA wherein the DNA encoding amino-acid racemase or D-alanine-D-alanine ligase is ligated to an expression vector, the transcription termination sequence is not essential, but it is preferred to place the transcription termination sequence immediately downstream of the structural gene.

Examples of such recombinant DNAs are pJBar1 and pEDd1B described below.

Examples of procaryotes include microorganisms belonging to the genera Escherichia, Serratia, Bacillus, Brevibacterium, Corynebacterium, Microbacterium, Pseudomonas, Agrobacterium, Alicyclobacillus, Anabaena, Anacystis, Arthrobacter, Azotobacter, Chromatium, Erwinia, Methylobacterium, Phormidium, Rhodobacter, Rhodopseudomonas, Rhodospirillum, Scenedesmus, Streptmyces, Synechoccus and Zymomonas. Specific examples are Escherichia coli XL1-Blue, Escherichia coli XL2-Blue, Escherichia coli DH1, Escherichia coli DH5a, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli MP347, Escherichia coli NM522, Escherichia coli BL21(DE3), Bacillus subtilis ATCC 33712, Bacillus megaterium, Bacillus sp. FERM BP-6030, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 14297, Corynebacterium acetoacidophilum ATCC 13870, Microbacterium ammoniaphilum ATCC 15354, Serratia ficaria, Serratia fonticola, Serratia liquefaciens, Serratia marcescens, Pseudomonas sp. D-0110, Agrobacterium radiobacter, Agrobacterium rhizogenes, Agrobacterium rubi, Anabaena cylindrica, Anabaena doliolum, Anabaena flos-aquae, Arthrobacter aurescens, Arthrobacter citreus, Arthrobacter globiformis, Arthrobacter hydrocarboglutamicus, Arthrobacter mysorens, Arthrobacter nicotianae, Arthrobacter paraffineus, Arthrobacter protophormiae, Arthrobacter roseoparaffinus, Arthrobacter sulfureus, Arthrobacter ureafaciens, Chromatium buderi, Chromatium tepidum, Chromatium vinosum, Chromatium warmingii, Chromatium fluviatile, Erwinia uredovora, Erwinia carotovora, Erwinia ananas, Erwinia herbicola, Erwinia punctata, Erwinia terreus, Methylobacterium rhodesianum, Methylobacterium extorguens, Phormidium sp. ATCC 29409, Rhodobacter capsulatus, Rhodobacter sphaeroides, Rhodopseudomonas blastica, Rhodopseudomonas marina, Rhodopseudomonas palustris, Rhodospirillum rubrum, Rhodospirillum salexigens, Rhodospirillum salinarum, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus and Zymomonas mobilis.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

5. Amino Acids Used as Substrates in the Process of the Present Invention

One or more kinds of amino acids selected from the group consisting of D-amino acids and glycine which are used as substrates in the process of the present invention may be any one or more kinds of amino acids selected from the group consisting of D-amino acids and glycine, provided that the one or more kinds of amino acids are not D-alanine alone or a combination of D-alanine and D-serine. Preferred are one or more kinds of amino acids selected from the group consisting of D-alanine, D-glutamine, D-glutamic acid, glycine, D-valine, D-leucine, D-isoleucine, D-proline, D-phenylalanine, D-tryptophan, D-methionine, D-serine, D-threonine, D-cysteine, D-asparagine, D-tyrosine, D-lysine, D-arginine, D-histidine, D-aspartic acid, D-ornithine and derivatives thereof, and preferred are one or more kinds of amino acids selected from the group consisting of D-alanine, glycine, D-leucine, D-isoleucine, D-proline, D-serine, D-threonine, D-cysteine and D-lysine. The above D-amino acids may be D-amino acids obtained by any methods, for example, commercially available D-amino acids, or D-amino acids produced by chemical synthesis or by using, as an enzyme source, a culture of a microorganism having the ability to produce D-amino acid or a treated matter of the culture.

The substance converted into D-amino acid by a culture of a microorganism having the ability to produce D-amino acid or a treated matter of the culture which is used as a substrate in the present invention may be any substance that is converted into D-amino acid by a culture of such microorganism or a treated matter thereof. For example, when a microorganism having the ability to produce hydantoinase is used as the microorganism having the ability to produce D-amino acid, one or more kinds of 5-substituted-DL-hydantoin, etc. are used. When a microorganism having the ability to produce D-aminoacylase is used, one or more kinds of N-acyl-DL-amino acids, etc. are used. When a microorganism having the ability to produce D-amino-acid amidase is used, one or more kinds of DL-amino acid amides, etc. are used. When a microorganism having the ability to produce D-amino-acid aminotransferase is used, one or more kinds of α-keto acids and one or more kinds of D-amino acids, etc. are used. When a microorganism having the ability to produce amino-acid racemase is used, one or more kinds of L-α-amino acids, etc. are used.

The above L-α-amino acids may be either natural or not, or may be derivatives thereof, so far as they can be used as substrates for amino-acid racemase.

Examples of the substrates for a microorganism having the ability to produce a protein having low-substrate-specific amino-acid racemase activity include one or more kinds of amino acids comprising one or more kinds of L-α-amino acids.

As the one or more kinds of amino acids comprising one or more kinds of L-α-amino acids, any combinations of any amino acids can be used so far as they are one ore more kinds of amino acids comprising one or more kinds of L-α-amino acids. Preferred are one or two kinds of amino acids comprising one or more kinds of L-α-amino acids selected from the group consisting of L-α-amino acids and glycine.

Examples of the L-α-amino acids are L-α-alanine, L-α-glutamine, L-α-glutamic acid, L-α-valine, L-α-leucine, L-α-isoleucine, L-α-proline, L-α-phenylalanine, L-α-tryptophan, L-α-methionine, L-α-serine, L-α-threonine, L-α-cysteine, L-α-asparagine, L-α-tyrosine, L-α-lysine, L-α-arginine, L-α-histidine, L-α-aspartic acid, L-α-ornithine and derivatives thereof.

The amino acids to be used in combination with the above L-α-amino acids may be any amino acids.

Examples of the amino acids are those selected from the group consisting of L- or D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid and ornithine, and glycine, and also include derivatives of these amino acids.

Examples of the derivatives of the above amino acids include hydroxyamino acids (e.g., β-hydroxyglutamine, β-hydroxyglutamic acid, γ-hydroxyglutamic acid, α-hydroxyglycine, β-hydroxyvaline, γ-hydroxyvaline, β-hydroxyleucine, γ-hydroxyleucine, δ-hydroxyleucine, β-hydroxyisoleucine, γ-hydroxyisoleucine, 3-hydroxyproline, 4-hydroxyproline, β-hydroxyphenylalanine, 3,4-dihydroxyphenylalanine, 2,4,5-trihydroxyphenylalanine, β-hydroxytryptophan, 5-hydroxytryptophan, α-hydroxymethionine, β-hydroxyserine, γ-hydroxythreonine, S-hydroxycysteine, β-hydroxyasparagine, β-hydroxytyrosine, β-hydroxylysine, γ-hydroxylysine, δ-hydroxylysine, N-hydroxylysine, β-hydroxyarginine, δ-hydroxyarginine, N-hydroxyarginine, β-hydroxyhistidine, β-hydroxyaspartic acid, β-hydroxyornithine, γ-hydroxyornithine and N-hydroxyornithine) and N-methyl amino acids (e.g., N-methyl-alanine, N-methyl-glutamine, N-methyl-glutamic acid, N-methyl-glycine, N-methyl-valine, N-methyl-leucine, N-methyl-isoleucine, N-methyl-proline, N-methyl-phenylalanine, N-methyl-tryptophan, N-methyl-methionine, N-methyl-serine, N-methyl-threonine, N-methyl-cysteine, N-methyl-asparagine, N-methyl-tyrosine, N-methyl-lysine, N-methyl-arginine, N-methyl-histidine, N-methyl-aspartic acid and N-methyl-ornithine).

6. Production Process of the Present Invention

The production process of the present invention relates to (1) a process for producing a dipeptide which comprises allowing an enzyme source, ATP and one or more kinds of amino acids selected from the group consisting of D-amino acids and glycine (provided that said one or more kinds of amino acids are not D-alanine alone or a combination of D-alanine and D-serine) to be present in an aqueous medium, said enzyme source being a culture of a microorganism having the ability to produce a protein having D-alanine-D-alanine ligase activity or a treated matter of the culture, allowing the dipeptide to form and accumulate in the aqueous medium, and recovering the dipeptide from the aqueous medium; and (2) a process for producing a dipeptide comprising D-amino acid which comprises allowing enzyme sources, ATP and a substance which is converted into the D-amino acid by a culture of a microorganism having the ability to produce the D-amino acid or a treated matter of the culture to be present in an aqueous medium, said enzyme sources being a culture of a microorganism having the ability to produce the D-amino acid or a treated matter of the culture, and a culture of a microorganism having the ability to produce a protein having D-alanine-D-alanine ligase activity or a treated matter of the culture, allowing the dipeptide comprising D-amino acid to form and accumulate in the aqueous medium, and recovering the dipeptide from the aqueous medium.

The enzyme source used in the process of the present invention can be obtained by culturing in a medium a microorganism having the ability to produce a protein having D-alanine-D-alanine ligase activity, a microorganism having the ability to produce D-amino acids or a microorganism having both the ability to produce a protein having D-alanine-D-alanine ligase activity and the ability to produce D-amino acid.

As the medium for culturing the microorganism, any of natural media and synthetic media can be used insofar as it is a medium suitable for efficient culturing of the microorganism which contains carbon sources, nitrogen sources, inorganic salts, etc. which can be assimilated by the microorganism.

As the carbon sources, any carbon sources that can be assimilated by the microorganism can be used. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

Examples of the nitrogen sources include ammonia, ammonium salts of organic or inorganic acids such as ammonium chloride, ammonium sulfate, ammonium acetate and ammonium phosphate, nitrogen-containing compounds, peptone, meat extract, yeast extract, corn steep liquor, casein hydrolyzate, soybean cake, soybean cake hydrolyzate, and various fermented microbial cells and digested products thereof.

Examples of the inorganic salts include potassium dihydrogenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration. The culturing temperature is preferably 15 to 60° C., and the culturing period is usually 5 hours to 7 days. The pH is maintained at 4 to 10 during the culturing. The pH adjustment is carried out by using an organic or inorganic acid, an alkali solution, urea, calcium carbonate, ammonia, etc.

If necessary, antibiotics such as ampicillin and tetracycline may be added to the medium during the culturing.

When a microorganism transformed with an expression vector comprising an inducible promoter is cultured, an inducer may be added to the medium, if necessary. For example, in the case of a microorganism transformed with an expression vector comprising lac promoter, isopropyl-β-D-thiogalactopyranoside or the like may be added to the medium; in the case of a microorganism transformed with an expression vector comprising trp promoter, indoleacrylic acid or the like may be added; and in the case of a microorganism transformed with an expression vector comprising Pm promoter, m-tolic acid may be added to the medium.

Examples of the treated matters of the culture include treated matters containing living cells such as concentrated culture, dried culture, cells obtained by centrifuging the culture, products obtained by subjecting the cells to drying, freeze-drying, treatment with a surfactant, treatment with a solvent and enzymatic treatment and a product obtained by subjecting the cells to immobilization; and products obtained by subjecting the cells to ultrasonication, mechanical friction and protein fractionation, as well as enzyme preparations obtained from the cells by extraction and purification.

The culture of a microorganism having D-alanine-D-alanine ligase activity or a treated matter thereof used in the above processes (1) and (2) is used at a concentration of 1 mU/l to 1000 U/l, preferably 10 mU/l to 100 U/l, one unit (U) being defined as the activity which forms 1 mmol of dipeptide comprising D-amino acid from one or two kinds of D-amino acids at 30° C. in one minute.

The culture of a microorganism having the ability to produce D-amino acid or a treated matter thereof used in the above process (2) is used at a concentration of 1 mU/l to 1000 U/l, preferably 10 mU/l to 100 U/l, one unit (U) being defined as the activity which forms 1 mmol of D-amino acid at 30° C. in one minute.

One or more kinds of amino acids selected from the group consisting of D-amino acids and glycine or the substance which is converted into D-amino acid by a culture of a microorganism having the ability to produce D-amino acid or a treated matter thereof used as a substrate is used at a concentration of 1 to 500 g/l in a total amount.

The aqueous media used in the above processes (1) and (2) include water, buffers such as phosphate buffer, carbonate buffer, acetate buffer, borate buffer, citrate buffer and Tris buffer, alcohols such as methanol and ethanol, esters such as ethyl acetate, ketones such as acetone, and amides such as acetamide. The culture of a microorganism used as the enzyme source can also be used as the aqueous medium.

In the above processes (1) and (2), a surfactant or an organic solvent may be added, if necessary. Any surfactant that does not prevent the dipeptide-forming reaction can be used. Suitable surfactants include nonionic surfactants such as polyoxyethylene octadecylamine (e.g., Nymeen S-215, NOF Corporation), cationic surfactants such as cetyltrimethylammonium bromide and alkyldimethylbenzylammonium chloride (e.g., Cation F2-40E, NOF Corporation), anionic surfactants such as lauroyl sarcosinate, and tertiary amines such as alkyldimethylamine (e.g., Tertiary Amine FB, NOF Corporation), which may be used alone or in combination. The surfactant is usually used at a concentration of 0.1 to 50 g/l. As the organic solvent, xylene, toluene, aliphatic alcohols, acetone, ethyl acetate, etc. may be used usually at a concentration of 0.1 to 50 ml/l.

The reactions of the above processes (1) and (2) are carried out in the aqueous medium at pH 5 to 10, preferably pH 6 to 9, at 20 to 60° C. for 1 to 96 hours.

The dipeptides produced by the above process (1) include dipeptides represented by the above formula (I) (wherein R¹ and R², which may be the same or different, each represent D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid or ornithine, or glycine, preferably D-form of alanine, leucine, isoleucine, proline, serine, threonine, cysteine or lysine, or glycine; provided that both R¹ and R² cannot represent D-alanine at the same time, and when R¹ is D-alanine, R² does not represent D-serine).

The dipeptides produced by the above process (2) include dipeptides represented by the above formula (II) (wherein R³ and R⁴, which may be the same or different, each represent L- or D-form of alanine, glutamine, glutamic acid, valine, leucine, isoleucine, proline, phenylalanine, tryptophan, methionine, serine, threonine, cysteine, asparagine, tyrosine., lysine, arginine, histidine, aspartic acid or ornithine, or glycine, preferably alanine, leucine, isoleucine, proline, serine, threonine, cysteine or lysine, or glycine; provided that both R³ and R⁴ cannot represent L-form of amino acids or glycine at the same time). More preferred examples of the dipeptides produced by process (2) are dipeptides represented by the above formula (III) (wherein R⁵ and R⁶, which may be the same or different, each represent D-alanine, D-glutamine, D-glutamic acid, D-valine, D-leucine, D-isoleucine, D-proline, D-phenylalanine, D-tryptophan, D-methionine, D-serine, D-threonine, D-cysteine, D-asparagine, D-tyrosine, D-lysine, D-arginine, D-histidine, D-aspartic acid, D-ornithine or glycine, preferably D-alanine, D-leucine, D-isoleucine, D-proline, D-serine, D-threonine, D-cysteine, D-lysine or glycine; provided that both R⁵ and R⁶ cannot represent glycine at the same time).

Recovery of the dipeptide formed in the aqueous medium can be carried out by ordinary methods of isolation and purification using active carbon, ion-exchange resins, etc.

Certain embodiments of the present invention are illustrated in the following examples. These examples are not to be construed as limiting the scope of the present invention.

In the following examples, analysis and determination of dipeptides and amino acids were carried out by the following method using high performance liquid chromatography (HPLC). The dipeptides and amino acids were analyzed by HPLC after being derivatized using (2,3-dinitro-5-fluorophenyl)-L-alaninamide (Na-FDAA). Derivatization by the use of FDAA was carried out by adding 50 μl of a 0.5% solution of Na-FDAA in acetone and 40 μl of 0.5 mol/l aqueous solution of sodium carbonate to 100 μl of a sample diluted with pure water, followed by stirring, and then allowing the resulting mixture to stand at 40° C. for 60 minutes.

To the above mixture were added 40 μl of 1 mol/l hydrochloric acid and 770 μl of methanol, followed by stirring. The resulting mixture was used as FDAA-derivatized sample.

The FDAA-derivatized sample was analyzed and determined using a WH-C18A column (Hitachi High-Tech Science Systems Corp., 4×150 mm). The following conditions were employed for HPLC analysis.

The following mobile phases were used:
mobile phase A consisting of 50 mmol/l potassium phosphate buffer (pH 2.7, adjusted with phosphoric acid), acetonitrile and methanol in 18:1:1 ratio;
mobile phase B consisting of 50 mmol/l potassium phosphate buffer (pH 2.7, adjusted with phosphoric acid), acetonitrile and methanol in 12:7:1 ratio; and
mobile phase C consisting of acetonitrile, tetrahydrofuran and methanol in 3:1:1 ratio.
The flow rate of the mobile phase was 0.5 ml/min, and the ratio of mobile phase A, mobile phase B and mobile phase C was changed as follows: minute 0 to 24, 100:0:0; minute 24 to 50, 55:45:0; minute 50 to 60, 0:100:0; minute 60 to 62, 0:0:100; and minute 62 to 80, 100:0:0.
Measurement of ultraviolet absorption was carried out at 340 nm at a column temperature of 40° C.

### EXAMPLE 1

### Construction of a Transformant Expressing D-Alanine-D-Alanine Ligase Gene ddlB Derived from Escherichia coli JM109

Escherichia coli JM109 was cultured in LB medium [10 g/l tryptone peptone (Difco), 10 g/l yeast extract (Difco) and 5 g/l sodium chloride] at 30° C. for 24 hours, and the culture was centrifuged to obtain cells.

The chromosomal DNA of the microorganism was isolated and purified from the cells by the method described in Current Protocols in Molecular Biology.

On the basis of the nucleotide sequence of D-alanine-D-alanine ligase gene ddlB of Escherichia coli K-12 (GenBank Accession No. NC_{—}000913), DNAs having the nucleotide sequences shown in SEQ ID NOS: 15 and 16 were synthesized using a DNA synthesizer (Model 8905, PerSeptive Biosystems, Inc.). PCR was carried out using the DNAs as a set of primers and the chromosomal DNA of Escherichia coli JM109 as a template. PCR was carried out using 50 μl of a reaction mixture comprising 0.1 μg of the chromosomal DNA, 0.5 μmol/l each of the primers, 2.5 units of ExTaq DNA polymerase (Takara Bio Inc.), 5 μl of buffer for ExTaq DNA polymerase (10×) and 200 μmol/l each of deoxyNTPs under the following conditions: incubation at 95° C. for 3 minutes; 25 cycles of 95° C. for 30 seconds., 45° C. for 30 seconds and 72° C. for one minute; and a final incubation at 72° C. for 5 minutes.

The resulting reaction mixture was mixed with an equal amount of phenol/chloroform (1 vol/1 vol) saturated with TE [10 mmol/l Tris-HCl, 1 mmol/l EDTA (pH 8.0)].

The resulting mixture was centrifuged, and the obtained upper layer was mixed with a two-fold volume of cold ethanol and allowed to stand at −80° C. for 30 minutes. The resulting solution was centrifuged, and the obtained DNA was dissolved in TE.

The DNA was digested with restriction enzymes NdeI and HindIII, and subjected to agarose gel electrophoresis to separate a ca. 0.9 kb DNA fragment. Then, the fragment was recovered from the gel and dissolved in TE. The obtained ca. 0.9 kb DNA fragment and NdeI-HindIII-digested pET21a(+) vector (Novagen, Inc.) obtained in the same manner as described above were subjected to ligation reaction using a ligation kit (Takara Bio Inc.) at 16° C. for one hour.

Escherichia coli JM109 was transformed using the reaction mixture according to the above known method, spread on LB agar medium (comprising 15 g/l agar in LB medium) containing 100 μg/ml ampicillin, and cultured overnight at 30° C.

A plasmid was extracted from a colony of the transformant that grew on the medium according to the method described in Molecular Biology, Third Edition, and its structure was analyzed using restriction enzymes. It was confirmed that plasmid pEDd1B expressing the ddlB gene having the nucleotide sequence shown in SEQ ID NO: 6 was obtained. Escherichia coli JM109 carrying pEDd1B was designated as Escherichia coli JM109/pEDd1B.

### EXAMPLE 2

### Construction of a Transformant Expressing D-Alanine-D-Alanine Ligase Gene ddlB Derived from Escherichia coli JM109 (2)

DNAs having the nucleotide sequences shown in SEQ ID NOS: 17 and 18 were synthesized. PCR was carried out using the DNAs as a set of primers and the chromosomal DNA of Escherichia coli JM109 prepared in Example 1 as a template. PCR was carried out using 50 μl of a reaction mixture comprising 0.1 μg of the chromosomal DNA, 0.5 μmol/l each of the primers, 2.5 units of KOD plus DNA polymerase (Toyobo Co., Ltd.), 5 μl of buffer for KOD plus DNA polymerase (10×) and 200 μmol/l each of deoxyNTPs under the following conditions: incubation at 94° C. for 2 minutes; 25 cycles of 94° C. for 30 seconds, 60° C. for 30 seconds and 68° C. for 90 seconds; and a final incubation at 68° C. for 4 minutes.

The resulting reaction mixture was mixed with an equal amount of TE, followed by centrifugation. The obtained upper layer was mixed with a two-fold volume of cold ethanol and allowed to stand at −80° C. for 30 minutes. The resulting solution was centrifuged, and the obtained DNA was dissolved in TE.

According to the protocol of Xa/LIC Cloning Kits (Novagen, Inc.), the DNA fragment amplified by PCR in the solution was inserted into pET-30 Xa/LIC vector (Novagen, Inc.).

Escherichia coli BL21(DE3) was transformed using the reaction mixture according to the above known method, spread on LB agar medium containing 50 μg/ml kanamycin, and cultured overnight at 30° C.

A plasmid was extracted from a colony of the transformant that grew on the medium according to the method described in Molecular Biology, Third Edition, and its structure was analyzed using restriction enzymes. It was confirmed that plasmid pEc-ddlB expressing the ddlB gene was obtained. Escherichia coli BL21(DE3) carrying pEc-ddlB was designated as Escherichia coli BL21(DE3)/pEc-ddlB.

### EXAMPLE 3

### Construction of a Transformant Expressing D-Alanine-D-Alanine Ligase Gene ddlA Derived from Escherichia coli JM109

On the basis of the nucleotide sequence of D-alanine-D-alanine ligase gene ddlA of Escherichia coli K-12 (GenBank Accession No. NC_{—}000913), DNAs having the nucleotide sequences shown in SEQ ID NOS: 19 and 20 were synthesized in the same manner as in Example 1. PCR was carried out using the DNAs as a set of primers and the chromosomal DNA of Escherichia coli JM109 prepared in Example 2 as a template. PCR was carried out using 50 μl of a reaction mixture comprising 0.1 μg of the chromosomal DNA, 0.5 μmol/l each of the primers, 2.5 units of KOD plus DNA polymerase (Toyobo Co., Ltd.), 5 μl of buffer for KOD plus DNA polymerase (10×) and 200 μmol/l each of deoxyNTPs under the following conditions: incubation at 94° C. for 2 minutes; 25 cycles of 94° C. for 30 seconds, 45° C. for 30 seconds and 68° C. for 90 seconds; and a final incubation at 68° C. for 4 minutes.

The DNA fragment amplified by PCR was ligated to pET-30 Xa/LIC vector in the same manner as in Example 2 to construct a recombinant DNA. Then, Escherichia coli BL21(DE3) was transformed using the recombinant DNA to obtain plasmid pEc-ddlA expressing the ddlA gene having the nucleotide sequence shown in SEQ ID NO: 7. Escherichia coli BL21(DE3) carrying pEc-ddlA was designated as Escherichia coli BL21(DE3)/pEc-ddlA.

### EXAMPLE 4

### Construction of a Transformant Expressing D-Alanine-D-Alanine Ligase Gene Derived from Oceanobacillus iheyensis HTE831

Oceanobacillus iheyensis HTE831 (purchased from RIKEN, Japan) was cultured in Modified Horikoshi-1 medium [10 g/l glucose, 5 g/l polypeptone (Nippon Shinyaku Co., Ltd.), 5 g/l yeast extract (Difco), 10 g/l sodium chloride, 1 g/l dipotassium hydrogenphosphate and 0.2 g/l magnesium sulfate heptahydrate, pH 9.0] at 30° C. for 24 hours, and the chromosomal DNA was prepared according to a known method.

On the basis of the nucleotide sequence of D-alanine-D-alanine ligase gene ddl of Oceanobacillus iheyensis HTE831 (described in DDBJ database: http://gib.genes.nig.ac.jp/single/index.php?spid=Oihe_HTE8 31), DNAs having the nucleotide sequences shown in SEQ ID NOS: 21 and 22 were synthesized. PCR was carried out using the DNAs as a set of primers and the chromosomal DNA of Oceanobacillus iheyensis HTE831 prepared above as a template. PCR was carried out using 50 μl of a reaction mixture comprising 0.1 μg of the chromosomal DNA, 0.5 μmol/l each of the primers, 2.5 units of KOD plus DNA polymerase, 5 μl of buffer for KOD plus DNA polymerase (10×) and 200 μmol/l each of deoxyNTPs under the following conditions: incubation at 94° C. for 2 minutes; 25 cycles of 94° C. for 30 seconds, 45° C. for 30 seconds and 68° C. for 90 seconds; and a final incubation at 68° C. for 4 minutes.

The DNA fragment amplified by PCR was ligated to pET-30 Xa/LIC vector in the same manner as in Example 3 to construct a recombinant DNA. Then, Escherichia coli BL21(DE3) was transformed using the recombinant DNA to obtain plasmid pOi-ddlA expressing the ddl gene having the nucleotide sequence shown in SEQ ID NO: 7. Escherichia coli BL21(DE3) carrying pOi-ddlA was designated as Escherichia coli BL21(DE3)/pOi-ddlA.

### EXAMPLE 5

### Construction of a Transformant Expressing D-Alanine-D-Alanine Ligase Gene Derived from Synechocystis sp. PPC6803

On the basis of the nucleotide sequence of D-alanine-D-alanine ligase gene ddl of Synechocystis sp. PPC6806 (described in DNA Data Bank of Japan (DDBJ) database: http://gib.genes.nig.ac.jp/single/index.php?spid=Syne_PCC6 803), DNAs having the nucleotide sequences shown in SEQ ID NOS: 23 and 24 were synthesized in the same manner as in Example 2. PCR was carried out using the DNAs as a set of primers and the chromosomal DNA of Synechocystis sp. PPC6806 (obtained from Kazusa DNA Research Institute) as a template. PCR was carried out using 50 μl of a reaction mixture comprising 0.1 μg of the chromosomal DNA, 0.5 μmol/l each of the primers, 2.5 units of KOD plus DNA polymerase, 5 μl of buffer for KOD plus DNA polymerase (10×) and 200 μmol/l each of deoxyNTPs under the following conditions: incubation at 94° C. for 2 minutes; 25 cycles of 94° C. for 30 seconds, 47° C. for 30 seconds and 68° C. for 90 seconds; and a final incubation at 68° C. for 4 minutes.

The DNA fragment amplified by PCR was ligated to pET-30 Xa/LIC vector in the same manner as in Example 2 to construct a recombinant DNA. Then, Escherichia coli BL21(DE3) was transformed using the recombinant DNA to obtain plasmid pSs-ddlA expressing the ddlA gene having the nucleotide sequence shown in SEQ ID NO: 9. Escherichia coli BL21(DE3) carrying pSs-ddlA was designated as Escherichia coli BL21(DE3)/pSs-ddlA.

### EXAMPLE 6

### Construction of a Transformant Expressing a Low-Substrate-Specific Amino-Acid Racemase Gene Derived from Pseudomonas putida IF012996

Pseudomonas putida IF012996 was cultured in an ordinary bouillon medium (Kyokuto Pharmaceutical Industrial Co., Ltd.) at 30° C. for 24 hours, and the culture was centrifuged to obtain cells.

The chromosomal DNA of the microorganism was isolated and purified from the cells by the method described in Current Protocols in Molecular Biology.

On the basis of the nucleotide sequence of a low-substrate-specific amino-acid racemase gene of Pseudomonas putida (WO03/074690), DNAs having the nucleotide sequences shown in SEQ ID NOS: 25 and 26 were synthesized using a DNA synthesizer (Model 8905, PerSeptive Biosystems, Inc.). PCR was carried out using the DNAs as a set of primers and the chromosomal DNA of Pseudomonas putida IF012996 as a template. PCR was carried out using 50 μl of a reaction mixture comprising 0.1 μg of the chromosomal DNA, 0.5 μmol/l each of the primers, 2.5 units of ExTaq DNA polymerase, 5 μl of buffer for ExTaq DNA polymerase (10×) and 200 μmol/l each of deoxyNTPs under the following conditions: incubation at 95° C. for 3 minutes; 25 cycles of 95° C. for 90 seconds, 60° C. for 60 seconds and 72° C. for 90 seconds; and a final incubation at 72° C. for 4 minutes.

The resulting reaction mixture was mixed with an equal amount of phenol/chloroform saturated with TE, followed by centrifugation. The obtained upper layer was mixed with a two-fold volume of cold ethanol and allowed to stand at −80° C. for 30 minutes. The resulting solution was centrifuged, and the obtained DNA was dissolved in 20 μl of TE.

The obtained DNA fragment was digested with restriction enzymes BamHI and EcoRI, and subjected to agarose gel electrophoresis to separate a ca. 1.2 kb DNA fragment. The fragment was recovered from the gel and dissolved in TE. Similarly, pSTV28 vector (Takara Bio Inc.) digested with BamHI and EcoRI was prepared. The ca. 1.2 kb DNA fragment and pSTV28 vector digested with the restriction enzymes were subjected to ligation reaction using a ligation kit at 16° C. for one hour.

Escherichia coli JM109 was transformed using the reaction mixture according to the above known method, spread on LB agar medium containing 25 μg/ml chloramphenicol, and cultured overnight at 30° C.

A plasmid was extracted from a colony of the transformant that grew on the medium according to the method described in Molecular Biology, Third Edition, and its structure was analyzed using restriction enzymes. It was confirmed that plasmid PSTBAR having DNA represented by the nucleotide sequence shown in SEQ ID NO: 13 was obtained.

On the basis of the nucleotide sequence of the low-substrate-specific amino-acid racemase gene of Pseudomonas putida (WO03/074690), DNAs having the nucleotide sequences shown in SEQ ID NOS: 27 and 28 were synthesized. PCR was carried out using the DNAs as a set of primers and the above-obtained PSTBAR as a template. PCR was carried out using 50 μl of a reaction mixture comprising 0.1 μg of the plasmid DNA, 0.5 μmol/l each of the primers, 2.5 units of ExTaq DNA polymerase, 5 μl of buffer for ExTaq DNA polymerase (10×) and 200 μmol/l each of deoxyNTPs under the following conditions: incubation at 95° C. for 3 minutes; 25 cycles of 95° C. for 30 seconds, 58° C. for 30 seconds and 72° C. for 90 seconds; and a final incubation at 72° C. for 4 minutes.

The resulting reaction mixture was mixed with an equal amount of phenol/chloroform saturated with TE, followed by centrifugation. The obtained upper layer was mixed with a two-fold volume of cold ethanol and allowed to stand at −80° C. for 30 minutes. The resulting solution was centrifuged, and the obtained DNA was dissolved in 20 μl of TE.

The obtained DNA fragment was partially digested with restriction enzymes AflIII and EcoRI, and subjected to agarose gel electrophoresis. A ca. 1.2 kb DNA fragment was recovered from the gel and dissolved in TE. The solution (5 μl) and pJB861 vector [Plasmid, 38, 35-51 (1997)] digested with AflIII and EcoRI were subjected to ligation reaction using a ligation kit at 16° C. for one hour.

Escherichia coli JM109 was transformed using the reaction mixture according to the above known method, spread on LB agar medium containing 50 μg/ml kanamycin, and cultured overnight at 30° C.

A plasmid was extracted from a colony of the transformant that grew on the medium according to the method described in Molecular Biology, Third Edition, and its structure was analyzed using restriction enzymes. It was confirmed that plasmid pJBar1 expressing the low-substrate-specific amino-acid racemase gene having the nucleotide sequence shown in SEQ ID NO: 13 was obtained. Escherichia coli JM109 carrying pJBar1 was designated as Escherichia coli JM109/pJBar1.

### EXAMPLE 7

### Preparation of D-Alanine-D-Alanine Ligase

Escherichia coli BL21(DE3) was transformed using the plasmid pEDd1B prepared in Example 1 according to the above known method, spread on LB agar medium containing 100 μg/ml ampicillin, and cultured overnight at 30° C.

Escherichia coli BL21(DE3) carrying pEDd1B was obtained from a colony of the transformant which grew and was designated as Escherichia coli BL21(DE3)/pEDd1B.

Escherichia coli BL21(DE3)/pEDd1B was cultured in LB medium containing 100 μg/ml ampicillin at 37° C. When OD (660 nm) reached 0.1, IPTG was added to the culture to give a concentration of 0.1 mmol/l, followed by further culturing for 2 hours. The resulting culture was centrifuged and the obtained cells were suspended in a 0.1 mol/l Tris-HCl buffer (pH 8.0). The suspension was subjected to ultrasonication and centrifugation to obtain the supernatant as a crude enzyme solution containing D-alanine-D-alanine ligase.

### EXAMPLE 8

### Production of D-Alanyl-D-Alanine Using D-Alanine as a Substrate

A reaction mixture (0.4 ml) comprising 0.1 mol/l Tris-HCl buffer (pH 7.5) containing the crude enzyme. solution containing D-alanine-D-alanine ligase prepared in Example 7 (protein amount: 0.4 mg/ml), 12.5 mmol/l D-alanine, 10 mmol/l ATP, 10 mmol/l magnesium chloride and 10 mmol/l potassium chloride was prepared and subjected to reaction at 37° C. for 5 hours. The reaction mixture was analyzed by HPLC, whereby 4.7 mmol/l D-alanyl-D-alanine was detected in the reaction mixture.

### EXAMPLE 9

### Production of D-Seryl-D-Serine Using D-Serine as a Substrate

A reaction mixture (0.4 ml) comprising 0.1 mol/l Tris-HCl buffer (pH 7.5) containing the crude enzyme solution containing D-alanine-D-alanine ligase prepared in Example 7 (protein amount: 0.4 mg/ml), 12.5 mmol/l D-serine, 10 mmol/l ATP, 10 mmol/l magnesium chloride and 10 mmol/l potassium chloride was prepared and subjected to reaction at 37° C. for 5 hours. The reaction mixture was analyzed by HPLC, whereby 4 mmol/l D-seryl-D-serine was detected in the reaction mixture.

### EXAMPLE 10

### Production of Glycyl-Glycine Using D-Alanine-D-Alanine Ligase

A reaction mixture (0.1 ml) comprising 0.1 mol/l Tris-HCl buffer (pH 7.5) containing the crude enzyme solution containing D-alanine-D-alanine ligase prepared in Example 7 (protein amount: 0.5 mg/ml), 40 mmol/l glycine, 20 mmol/l ATP, 20 mmol/l magnesium chloride and 10 mmol/l potassium chloride was prepared and subjected to reaction at 37° C. for 24 hours. The reaction mixture was analyzed by HPLC, whereby 4 mmol/l glycyl-glycine was detected in the reaction mixture.

### EXAMPLE 11

### Production of D-Cysteinyl-D-Cysteine Using D-Cysteine as a Substrate

A reaction mixture (0.1 ml) comprising 0.1 mol/l Tris-HCl buffer (pH 7.5) containing the crude enzyme solution containing D-alanine-D-alanine ligase prepared in Example 7 (protein amount: 0.5 mg/ml), 40 mmol/l D-cysteine, 20 mmol/l ATP, 20 mmol/l magnesium chloride and 10 mmol/l potassium chloride was prepared and subjected to reaction at 37° C. for 24 hours. The reaction mixture was analyzed by HPLC, whereby 3 mmol/l D-cysteinyl-D-cysteine was detected in the reaction mixture.

### EXAMPLE 12

### Production of D-Threonyl-D-Threonine Using D-Threonine as a Substrate

A reaction mixture (0.1 ml) comprising 0.1 mol/l Tris-HCl buffer (pH 7.5) containing the crude enzyme solution containing D-alanine-D-alanine ligase prepared in Example 7 (protein amount: 0.5 mg/ml), 40 mmol/l D-threonine, 20 mmol/l ATP, 20 mmol/l magnesium chloride and 10 mmol/l potassium chloride was prepared and subjected to reaction at 37° C. for 24 hours. The reaction mixture was analyzed by HPLC, whereby 1 mmol/l D-threonyl-D-threonine was detected in the reaction mixture.

### EXAMPLE 13

### Preparation of D-Alanine-D-Alanine Ligase and Low-Substrate-Specific Amino-Acid Racemase

Escherichia coli BL21(DE3)/pEDd1B prepared in Example 6 was transformed using the plasmid pJBar1 prepared in Example 6 according to the above known method, spread on LB agar medium containing 100 μg/ml ampicillin and 50 μg/ml kanamycin, and cultured overnight at 30° C.

A transformant carrying two plasmid DNAs (pEDd1B and pJBar1) was obtained from a colony of the transformant that grew on the medium and was designated as Escherichia coli BL21(DE3)/pJBar1,pEDd1B.

Escherichia coli BL21(DE3)/pJBar1,pEDd1B was cultured in LB medium containing 100 μg/ml ampicillin and 50 μg/ml kanamycin at 37° C. When OD (660 nm) reached 0.1, IPTG and m-tolic acid were added to the culture to give concentrations of 0.1 mmol/l and 3 mmol/l, respectively, followed by further culturing for 2 hours. The resulting culture was centrifuged and the obtained cells were suspended in a 0.1 mol/l Tris-HCl buffer (pH 8.0). The suspension was subjected to ultrasonication and centrifugation to obtain the supernatant as a crude enzyme solution containing D-alanine-D-alanine ligase and amino-acid racemase.

### EXAMPLE 14

### Production of D-Alanyl-D-Alanine Using L-Alanine as a Substrate

A reaction mixture (0.4 ml) comprising 0.1 mol/l Tris-HCl buffer (pH 7.5) containing the crude enzyme solution containing D-alanine-D-alanine ligase and amino-acid racemase prepared in Example 13 (protein amount: 0.4 mg/ml), 20 mmol/l L-alanine, 10 mmol/l ATP, 10 mmol/l magnesium chloride and 10 mmol/l potassium chloride was prepared and subjected to reaction at 37° C. for 3 hours. The reaction mixture was analyzed by HPLC, whereby 6.8 mmol/l D-alanyl-D-alanine was detected in the reaction mixture.

### EXAMPLE 15

### Production of D-Seryl-D-Serine Using L-Serine as a Substrate

A reaction mixture (0.4 ml) comprising 0.1 mol/l Tris-HCl buffer (pH 7.5) containing the crude enzyme solution containing D-alanine-D-alanine ligase and amino-acid racemase prepared in Example 13 (protein amount: 0.4 mg/ml), 20 mmol/l L-serine, 10 mmol/l ATP, 10 mmol/l magnesium chloride and 10 mmol/l potassium chloride was prepared and subjected to reaction at 37° C. for 3 hours. The reaction mixture was analyzed by HPLC, whereby 3 mmol/l D-seryl-D-serine was detected in the reaction mixture.

### EXAMPLE 16

### Production of D-Alanyl-D-Serine Using L-Alanine and L-Serine as Substrates

A reaction mixture (0.4 ml) comprising 0.1 mol/l Tris-HCl buffer (pH 7.5) containing the crude enzyme solution containing D-alanine-D-alanine ligase and amino-acid racemase prepared in Example 13 (protein amount: 0.4 mg/ml), 10 mmol/l L-alanine, 10 mmol/l L-serine, 10 mmol/l ATP, 10 mmol/l magnesium chloride and 10 mmol/l potassium chloride was prepared and subjected to reaction at 37° C. for 3 hours. The reaction mixture was analyzed by HPLC, whereby 4.5 mmol/l D-alanyl-D-serine and 2 mmol/l D-alanyl-D-alanine were detected in the reaction mixture.

### EXAMPLE 17

### Construction of a Transformant Expressing a D-Alanine-D-Alanine Ligase Gene Derived from Thermotoga maritima ATCC 43589

On the basis of the nucleotide sequence of a D-alanine-D-alanine ligase gene of Thermotoga maritima ATCC 43589 [described in DNA Data Bank of Japan (DDBJ) database: http://gib.genes.nig.ac.jp/common/info.php?spid=Tmar_MSB8& ftid=267], DNAs having the nucleotide sequences shown in SEQ ID NOS: 29 and 30 were synthesized in the same manner as in Example 2. PCR was carried out using the DNAs as a set of primers and the chromosomal DNA of Thermotoga maritima ATCC 43589 as a template. PCR was carried out using 50 μl of a reaction mixture comprising 0.1 μg of the chromosomal DNA, 0.5 μmol/l each of the primers, 2.5 units of KOD plus DNA polymerase, 5 μl of buffer for KOD plus DNA polymerase (10×) and 200 μmol/l each of deoxyNTPs under the following conditions: incubation at 94° C. for 2 minutes; 25 cycles of 94° C. for 30 seconds, 47° C. for 30 seconds and 68° C. for 90 seconds; and a final incubation at 68° C. for 4 minutes.

The DNA fragment amplified by PCR was ligated to pET-30 Xa/LIC vector in the same manner as in Example 2 to construct a recombinant DNA. Then, Escherichia coli BL21(DE3) was transformed using the recombinant DNA to obtain plasmid pTmDd1 expressing the D-alanine-D-alanine ligase gene having the nucleotide sequence shown in SEQ ID NO: 10. Escherichia coli BL21(DE3) carrying pTmDd1 was designated as Escherichia coli BL21(DE3)/pTmDd1.

### EXAMPLE 18

### Production of D-Amino Acid-Comprising Dipeptides Using D-Alanine-D-Alanine Ligase of Thermotoga maritima ATCC 43589

A crude enzyme solution containing D-alanine-D-alanine ligase derived from Thermotoga maritima ATCC 43589 was prepared from Escherichia coli BL21(DE3)/pTmDd1 in the same manner as in Example 7. A reaction mixture (0.1 ml) comprising 0.1 mol/l Tris-HCl buffer (pH 7.5) containing the crude enzyme solution (protein amount: 0.5 mg/ml), 40 mmol/l D-alanine, 20 mmol/l ATP, 20 mmol/l magnesium chloride and 10 mmol/l potassium chloride was prepared and subjected to reaction at 37° C. for 5 hours. The reaction mixture was analyzed by HPLC, whereby 11 mmol/l D-alanyl-D-alanine was detected in the reaction mixture. When D-serine was used in place of D-alanine in the above reaction, 9 mmol/l D-seryl-D-serine was detected; when D-cysteine was used, 9 mmol/l D-cysteinyl-D-cysteine was detected; when D-threonine was used, 4 mmol/l D-threonyl-D-threonine was detected; and when glycine was used, 8 mmol/l glycyl-glycine was detected.

### INDUSTRIAL APPLICABILITY

The present invention provides an efficient process for producing various kinds of dipeptides comprising D-amino acids.

Sequence Listing Free Text

SEQ ID NO: 15—Description of Artificial Sequence: Synthetic DNA
SEQ ID NO: 16—Description of Artificial Sequence: Synthetic DNA
SEQ ID NO: 17—Description of Artificial Sequence: Synthetic DNA
SEQ ID NO: 18—Description of Artificial Sequence: Synthetic DNA
SEQ ID NO: 19—Description of Artificial Sequence: Synthetic DNA
SEQ ID NO: 20—Description of Artificial Sequence: Synthetic DNA
SEQ ID NO: 21—Description of Artificial Sequence: Synthetic DNA
SEQ ID NO: 22—Description of Artificial Sequence: Synthetic DNA
SEQ ID NO: 23—Description of Artificial Sequence: Synthetic DNA
SEQ ID NO: 24—Description of Artificial Sequence: Synthetic DNA
SEQ ID NO: 25—Description of Artificial Sequence: Synthetic DNA
SEQ ID NO: 26—Description of Artificial Sequence: Synthetic DNA
SEQ ID NO: 27—Description of Artificial Sequence: Synthetic DNA
SEQ ID NO: 28—Description of Artificial Sequence: Synthetic DNA
SEQ ID NO: 29—Description of Artificial Sequence: Synthetic DNA
SEQ ID NO: 30—Description of Artificial Sequence: Synthetic DNA

1. A process for producing a dipeptide, which comprises: allowing an enzyme source, ATP and one or more kinds of amino acids selected from the group consisting of D-amino acids and glycine (provided that said one or more kinds of amino acids are not D-alanine alone or a combination of D-alanine and D-serine) to be present in an aqueous medium, said enzyme source being a culture of a microorganism having the ability to produce a protein having D-alanine-D-alanine ligase activity or a treated matter of the culture; allowing the dipeptide to form and accumulate in the aqueous medium; and recovering the dipeptide from the aqueous medium.

2. The process according to claim 1, wherein the D-amino acid is produced by using a culture of a microorganism having the ability to produce the D-amino acid or a treated matter of the culture as an enzyme source.

3. A process for producing a dipeptide comprising D-amino acid which comprises: allowing enzyme sources, ATP and a substance which is converted into the D-amino acid by a culture of a microorganism having the ability to produce the D-amino acid or a treated matter of the culture to be present in an aqueous medium, said enzyme sources being a culture of a microorganism having the ability to produce the D-amino acid or a treated matter of the culture, and a culture of a microorganism having the ability to produce a protein having D-alanine-D-alanine ligase activity or a treated matter of the culture; allowing the dipeptide comprising D-amino acid to form and accumulate in the aqueous medium; and recovering the dipeptide from the aqueous medium.

4. The process according to claim 1, wherein the protein having D-alanine-D-alanine ligase activity is a protein selected from the group consisting of the following [1] to [3]:
[1] a protein having the amino acid sequence shown in any of
SEQ ID NOS: 1 to 5;
[2] a protein consisting of an amino acid sequence wherein one or more amino acid residues are deleted, substituted or added in the amino acid sequence shown in any of SEQ ID NOS: 1 to 5 and having D-alanine-D-alanine ligase activity; and
[3] a protein consisting of an amino acid sequence which has 80% or more homology to the amino acid sequence shown in any of SEQ ID NOS: 1 to 5 and having D-alanine-D-alanine ligase activity.

5. The process according to claim 1, wherein the microorganism having the ability to produce a protein having D-alanine-D-alanine ligase activity is a microorganism obtainable by introducing DNA encoding the protein having D-alanine-D-alanine ligase activity into a host cell.

6. The process according to claim 5, wherein the DNA encoding a protein having D-alanine-D-alanine ligase activity is DNA selected from the group consisting of the following [1] to [4]:
[1] DNA encoding a protein having the amino acid sequence shown in any of SEQ ID NOS: 1 to 5;
[2] DNA having the nucleotide sequence shown in any of SEQ ID NOS: 6 to 10;
[3] DNA which hybridizes with DNA having a nucleotide sequence complementary to the nucleotide sequence shown in any of SEQ ID NOS: 6 to 10 under stringent conditions and which encodes a protein having D-alanine-D-alanine ligase activity; and
[4] DNA consisting of a nucleotide sequence which has 90% or more homology to the nucleotide sequence shown in any of SEQ ID NOS: 6 to 10 and encoding a protein having D-alanine-D-alanine ligase activity.

7. The process according to claim 2, wherein the microorganism having the ability to produce D-amino acid is a microorganism having the ability to produce a protein having the activity of hydantoinase, D-aminoacylase, D-amino-acid amidase, D-amino-acid transaminase or amino-acid racemase.

8. The process according to claim 7, wherein the protein having amino-acid racemase activity is a protein having low-substrate-specific amino-acid racemase activity.

9. The process according to claim 8, wherein the protein having low-substrate-specific amino-acid racemase activity is a protein selected from the group consisting of the following [1] to [3]:
[1] a protein having the amino acid sequence shown in SEQ ID NO: 11 or 12;
[2] a protein consisting of an amino acid sequence wherein one or more amino acid residues are deleted, substituted or added in the amino acid sequence shown in SEQ ID NO: 11 or 12 and having low-substrate-specific amino-acid racemase activity; and
[3] a protein consisting of an amino acid sequence which has 80% or more homology to the amino acid sequence shown in SEQ ID NO: 11 or 12 and having low-substrate-specific amino-acid racemase activity.

10. The process according to claim 2, wherein the microorganism having the ability to produce D-amino acid is a microorganism obtainable by introducing DNA encoding a protein having the activity of hydantoinase, D-aminoacylase, D-amino-acid am idase, D-amino-acid am inotransferase or amino-acid racemase into a host cell.

11. The process according to claim 10, wherein the DNA encoding a protein having amino-acid racemase activity is DNA encoding low-substrate-specific amino-acid racemase.

12. The process according to claim 11, wherein the DNA encoding a protein having low-substrate-specific amino-acid racemase activity is DNA selected from the group consisting of the following [1] to [4]:
[1] DNA encoding a protein having the amino acid sequence shown in SEQ ID NO: 11 or 12;
[2] DNA having the nucleotide sequence shown in SEQ ID NO: 13 or 14;
[3] DNA which hybridizes with DNA having a nucleotide sequence complementary to the nucleotide sequence shown in SEQ ID NO: 13 or 14 under stringent conditions and which encodes a protein having low-substrate-specific amino-acid racemase activity; and
[4] DNA consisting of a nucleotide sequence which has 90% or more homology to the nucleotide sequence shown in SEQ ID NO: 13 or 14 and encoding a protein having low-substrate-specific amino-acid racemase activity.

13. The process according to claim 1, wherein the one or more kinds of amino acids selected from the group consisting of D-amino acids and glycine are amino acids selected from the group consisting of D-alanine, D-glutamine, D-glutamic acid, glycine, D-valine, D-leucine, D-isoleucine, D-proline, D-phenylalanine, D-tryptophan, D-methionine, D-serine, D-threonine, D-cysteine, D-asparagine, D-tyrosine, D-lysine, D-arginine, D-histidine, D-aspartic acid and D-ornithine (provided that said one or more kinds of amino acids are not D-alanine alone or a combination of D-alanine and D-serine).

14. The process according to claim 3, wherein the substance which is converted into D-amino acid by a culture of a microorganism having the ability to produce the D-amino acid or a treated matter of the culture is one or more kinds of 5-substituted-DL-hydantoin, one or more kinds of N-acyl-DL-amino acids, one or more kinds of DL-amino acid amides, one or more kinds of α-keto acids and one or more kinds of D-amino acids, or one or more kinds of amino acids comprising one or more kinds of L-α-amino acids.

15. The process according to claim 14, wherein the one or more kinds of amino acids comprising one or more kinds of L-α-amino acids comprise one or more kinds of amino acids selected from the group consisting of L- or D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid and ornithine, and glycine.

16. The process according to claim 1, wherein the dipeptide is a dipeptide represented by formula (I):
R¹—R² (I)
(wherein R¹ and R², which may be the same or different, each represent D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid or ornithine, or glycine; provided that both R¹ and R² cannot represent D-alanine at the same time, and when R¹ is D-alanine, R² does not represent D-serine).

17. The process according to claim 3, wherein the dipeptide comprising D-amino acid is a dipeptide represented by formula (II):
R³—R⁴ (II)
(wherein R³ and R⁴, which may be the same or different, each represent L- or D-form of alanine, glutamine, glutamic acid, valine, leucine, isoleucine, proline, phenylalanine, tryptophan, methionine, serine, threonine, cysteine, asparagine, tyrosine, lysine, arginine, histidine, aspartic acid or ornithine, or glycine; provided that both R³ and R⁴ cannot represent L-form of amino acids or glycine at the same time).

18. The process according to claim 3, wherein the dipeptide comprising D-amino acid is a dipeptide represented by formula (III):
R⁵—R⁶ (III)
(wherein R⁵ and R⁶, which may be the same or different, each represent D-alanine, D-glutamine, D-glutamic acid, D-valine, D-leucine, D-isoleucine, D-proline, D-phenylalanine, D-tryptophan, D-methionine, D-serine, D-threonine, D-cysteine, D-asparagine, D-tyrosine, D-lysine, D-arginine, D-histidine, D-aspartic acid, D-ornithine or glycine; provided that both R⁵ and R⁶ cannot represent glycine at the same time).


SEQUENCE LISTING (30 sequences)

SEQ ID NO: 1 - protein, 306 aa, Escherichia coli K-12 [sequence omitted]

SEQ ID NO: 2 - protein, 364 aa, Escherichia coli K-12
MEKLRVGIVFGGKSAEHEVSLQSAKNIVDAIDKSRFDVVLLGIDKQGQWHVSDASNYLLNADDPAHIALRPSATSLAQVPGKHEHQLIDAQNGQPLPTVDVIFPIVHGTLGEDGSLQGMLRVANLPFVGSDVLASAACMDKDVTKRLLRDAGLNIAPFITLTRANRHNISFAEVESKLGLPLFVKPANQGSSVGVSKVTSEEQYAIAVDLAFEFDHKVIVEQGIKGREIECAVLGNDNPQASTCGEIVLTSDFYAYDTKYIDEDGAKVVVPAAIAPEINDKIRAIAVQAYQTLGCAGMARVDVFLTPENEVVINEINTLPGFTNISMYPKLWQASGLGYTDLITRLIELALERHAADNALKTTM

SEQ ID NO: 3 - protein, 354 aa, Oceanobacillus oheyensis HTE831
MRVGLLFGGCSGEHEVSIKSAQAIAKALASDDNQTKYQVSPFYIQKNGVWLGPDVSQQVLDQGVPWGDQPVTAGQRWQFPPEAARMEVWFPILHGPNGEDGTVQGLFSLMQVPYVGSGVLGSCVGMDKLAMKMVFERAGLPQVNYMGVERGEIWSNPCVFPALCEKIEAQVGYPCFVKPANLGSSVGIAKVRNRSELEAALDNAASYDRRIIVEAGLTDIREVECAVLGNENPQASVVGEITYDSDFYDYETKYTDGRSQMHIPANLPKAVVNQIQTMAIQAFKAVDAAGLGRLDFFYQPTTGQIVINEINTLPGFTAFSMYPELWRASGVEFPSLVDRLLQLALDYHGRPGHQ

SEQ ID NO: 4 - protein, 366 aa, Synechocystis sp. PPC6806
MKKKVGIVFGGKSAEHEVSLQSAKNIVDAIDSDKYDVYLLGIDKQGNWHVNDRSHYLINEEDPKLISLNKTNEGIAVIPGAEKNQIVTSNSTKSLDQLDVIFPIVHGTLGEDGSLQGMLRMANIPFVGSSVLGSAISMDKDIAKRLLKDAGLKVAKGYAYRSVDKESIHFDSLKEELGLPMFIKPANQGSSVGVHKVENEEQFYSAIKDAFQFDHKLLVEEAIVGREIECAVLGNEKPAASVPGEILPTGDFYSYEAKYIDETGAVLQIPAKLEEPVVDSIQDIALQAFKALNCEGLARVDVFLTDDNEIVINEINTLPGFTKISMYPKLWEESGVSYRELIENLIELAIERNQRDQQLKSSRSDG

SEQ ID NO: 5 - protein, 303 aa, Thermotoga maritima ATCC 43589
MRVALLMGGVSREREISLRSGERVKKALEKLGYEHTVFDVREDFLKKVDQLKSFDVVFNVLHGTFGEDGTLQAILDFLGIRYTGSDAFSSMICFDKLVTYRFLKGTVEIPDFVEIKEFMKTSPLGYPCVVKPRREGSSIGVFVCESDEEFQHALKEDLPRYGSVIVQKYIPGREMTVSILETEKGFEILPVLELRPKRRFYDYVAKYTKGETEFILPAPLNPSEERLVKEMALKAFVEAGCRGFGRVDGIFSDGRFYFLEINTVPGLTETSDLPASAKAGGIEFEELVEIILKSAFLKEGVRV

SEQ ID NO: 6 - DNA, 918 bp, Escherichia coli K-12
atgactgataaaatcgcggtcctgttgggtgggacctccgctgagcgggaagtttctctgaattctggcgcagcggtgttagccggactgcgtgaaggcggtattgacgcgtatcctgtcgacccgaaagaagtcgacgtgacgcaactgaagtcgatgggctttcagaaagtgtttatcgcgctacacggtcgcggcggtgaagatggtacgctgcaggggatgctcgagctgatgggcttgccttataccggaagcggagtgatggcatctgcgctttcaatggataaactacgcagcaaacttctatggcaaggtgccggtttaccggtcgcgccgtgggtagcgttaacccgcgcagagtttgaaaaaggcctgagcgataagcagttagcagaaatttctgctctgggtttgccggttatcgttaagccgagccgcgaaggttccagtgtgggaatgtcaaaagtagtagcagaaaatgctctacaagatgcattaagattggcatttcagcacgatgaagaagtattgattgaaaaatggctaagtgggccggagttcacggttgcgatactcggtgaagaaattttaccgtcaatacgtattcaaccgtccggaaccttctatgattatgaggcgaagtatctctctgatgagacacagtatttctgccccgcaggtctggaagcgtcacaagaggccaatttgcaggcattagtgctgaaagcatggacgacgttaggttgcaaaggatggggacgtattgacgttatgctggacagcgatggacagttttatctgctggaagccaatacctcaccgggtatgaccagccacagcctggtgccgatggcggcacgtcaggcaggtatgagcttctcgcagttggtagtacgaattctggaactggcggac

SEQ ID NO: 7 - DNA, 1092 bp, Escherichia coli K-12
atggaaaaactgcgggtaggaatcgtttttggtggtaaatcagcggaacatgaagtgtctctgcaatcggcaaaaaacattgtcgatgccattgataaaagtcgcttcgacgttgtgctgctgggcattgataaacaagggcaatggcacgtcagcgatgccagcaattatctgctaaatgcagacgatcctgcccatattgcgttgcgcccttcggcgaccagccttgcgcaggtgccaggtaaacatgagcatcagcttatcgacgcgcaaaacggtcagccgttgccgacggtggatgtcattttcccgattgtccacggtacgctgggcgaagatggttccttgcagggaatgctgcgggtcgccaatttaccgtttgtaggttctgatgttctggcttcagcagcctgtatggacaaagatgtcaccaaacgtctgctacgcgatgccgggctgaacattgcgccatttattaccctgacgcgcgccaatcgtcacaacattagttttgccgaagtggagtctaaactggggttaccactgtttgtaaaaccggctaatcagggctcttctgttggtgtcagcaaagtaaccagtgaagaacagtacgcaattgccgtcgatctggcgttcgagttcgatcataaagtgatcgttgagcaagggatcaaaggtcgtgagatcgaatgcgcagttctgggcaacgacaatccgcaagccagcacctgtggagagatcgtactcaccagcgatttctatgcctacgacaccaagtacattgacgaagatggcgcgaaagtggtagttccggcagccattgcgccagaaatcaacgataagatccgggcgattgccgttcaggcttatcaaacgttgggatgcgcaggcatggcgcgtgtagacgtgtttttaaccccagagaacgaagtggtgatcaacgagatcaacaccctgcctggcttcactaatatcagtatgtatccgaagctgtggcaagccagcggtctgggttacaccgatctgatcacacgtttgattgaactggcgctggagcgtcacgctgcggataacgcattgaaaaccacaatg

SEQ ID NO: 8 - DNA, 1062 bp, Oceanobacillus oheyensis HTE831
atgcgtgtggggctgttatttggtgggtgttctggggagcatgaagtctcgatcaaatccgcccaggcgatcgccaaagcattggccagcgacgataaccaaaccaaatatcaagtaagccctttttatattcaaaaaaatggcgtttggctggggccagacgtttctcaacaggttttggaccagggagttccctggggggatcagcccgttacggccggacaacggtggcagtttccccccgaagcggcccgaatggaggtgtggtttcccattttgcacggccccaacggtgaagatggcacagtgcaggggttatttagtttgatgcaagtgccctatgtgggcagtggcgtgctgggttcctgtgtgggcatggataaattggcgatgaaaatggtctttgaacgggcagggttgccccaggtcaattacatgggggtagaacggggggaaatttggtctaatccctgtgtttttccggctttgtgcgaaaaaattgaagcccaggtgggctatccctgtttcgttaaaccggccaacttaggctcctccgtgggcattgccaaagtgcggaaccgtagtgaattggaagcagccctagacaacgccgccagttacgatcgccgcatcattgtcgaagcgggtttaacggatattagggaagtggaatgtgcggtgctgggcaacgaaaatccccaagcttccgtggtgggggaaatcacctacgacagcgacttttacgattacgaaaccaaatacaccgatggccgctcccaaatgcatatccccgccaacttacctaaagctgtggtgaaccagatccagaccatggccattcaagcttttaaagctgtggatgcggcgggcttaggacgattggactttttctatcaaccgactacggggcagattgtcatcaacgaaattaacaccctgcctggtttcaccgcctttagtatgtatcccgaactgtggcgagcttctggggtagaatttcctagcttagtggatcgattattacagttggcattggactaccatggccgtcccggtcaccaa

SEQ ID NO: 9 - DNA, 1098 bp, Synechocystis sp. PPC6806
atgaagaaaaaagttggtattgtatttggaggtaagtcagcagaacatgaggtttcactgcagtctgctaaaaacattgtagatgcaatcgactcagataaatacgacgtatacttattaggaattgataagcaagggaattggcatgtcaatgatcgatcgcattacttgataaatgaagaggatccgaaattaatttcattaaacaaaacaaatgaaggaattgctgttattcctggagcagaaaaaaaccagatcgttacttcgaattcaactaaatctcttgatcagttagatgtaatttttccaattgttcatgggacgctcggagaagatggaagcttacaagggatgcttcgaatggcaaacattccatttgtcggatctagtgtattgggctctgctattagcatggataaagacattgccaaacgtctattgaaagacgctggattaaaagtagcaaaaggatatgcttatcgaagtgtggacaaggaaagcattcattttgatagtcttaaagaagaattaggattacctatgtttattaaaccagcaaaccagggttcgtcggttggtgttcacaaagtagaaaacgaagaacaattttattcggctattaaggatgcatttcaatttgatcataaattactcgttgaagaagcaattgtaggaagggaaattgaatgtgctgttctgggaaatgaaaaaccagctgcatctgttcctggagaaattttaccgacgggagatttttactcctatgaagcgaaatatattgatgaaacgggggcagtacttcaaattcctgctaaactagaagaacctgtagtagacagtatacaagatattgctttacaagcttttaaggcattgaattgtgaaggtctggcaagagttgatgtgtttttaacggatgacaacgaaattgtaatcaatgaaataaatacacttccaggttttacaaaaataagcatgtatccaaaattatgggaagaaagcggagtatcttatcgtgaattaatcgagaatttaatagaacttgctatagagagaaatcaacgtgaccaacaattaaagagttctagaagtgatgga

SEQ ID NO: 10 - DNA, 909 bp, Thermotoga maritima ATCC 43589
gtgagagtggctctcctgatgggaggagtgtcgagggaaagggaaatctctctgagaagcggagaaagggtaaaaaaggctcttgaaaaactcgggtatgaacacaccgtttttgacgttagagaagattttctcaagaaagtggatcagttgaaaagttttgatgtggtcttcaacgttctccatggaactttcggtgaggatggaacgcttcaggcaattctggattttttggggatcagatacaccgggtccgatgcgttttcaagcatgatatgttttgataaactcgtgacttacaggtttttaaagggcaccgtcgagatacctgattttgtggagatcaaggaattcatgaaaacttctccccttgggtatccctgtgtggtaaaaccgaggagagagggttcaagcataggagtgttcgtttgtgaatcggatgaagagtttcaacacgcgctcaaggaagatttaccacgatatgggagtgtgattgttcaaaagtacatccccggcagggaaatgactgtgtctattttagaaacggaaaagggcttcgaaattcttcccgttcttgagttgcgaccaaagcgtcgtttctacgattacgttgcgaagtacacgaaaggtgaaacggagttcattcttcctgctcctttgaatccttcagaagaacgccttgtcaaggaaatggctctcaaagcgtttgtagaagctggttgcagaggttttgggagggtggatggtattttcagcgatggcaggttctactttctggaaatcaacacggtaccgggtctaacggaaaccagtgatctcccagcgagcgcgaaagcgggaggaatcgagttcgaggaactcgttgagatcatcttaaagagcgcttttttgaaagaaggggtgagagta

SEQ ID NO: 11 - protein, 409 aa, Pseudomonas putida KT2440(ATCC47954)
MPFRRTLLAASLALLITGQAPLYAAPPLSMDNGTNTLTVQNSNAWVEVSASALQHNIRTLQAELAGKSKLCAVLKADAYGHGIGLVMPSIIAQGVPCVAVASNEEARVVRASGFTGQLVRVRLASLSELEDGLQYDMEELVGSAEFARQADAIAARHGKTLRIHMALNSSGMSRNGVEMATWSGRGEALQITDQKHLKLVALMTHFAVEDKDDVRKGLAAFNEQTDWLIKHARLDRSKLTLHAANSFATLEVPEARLDMVRTGGALFGDTVPARTEYKRAMQFKSHVAAVHSYPAGNTVGYDRTFTLARDSRLANITVGYSDGYRRVFTNKGHVLINGHRVPVVGKVSMNTLMVDVTDFPDVKGGNEVVLFGKQAGGEITQAEMEEINGALLADLYTVWGNSNPKILVD

SEQ ID NO: 12 - protein, 409 aa, Pseudomonas putida IFO12996
MQFSRTLLAASLALLITGQAPLYAAPPLSMDNGTTALTAQNSNAWVEISAGALQHNIRTLQAELGGKSKLCAVLKADAYGHGIGLVMPSIIAQGVPCVAVASNEEARVVRASGFTGQLVRVRLASLGEVEDALQYDMEELVGSAEFARQLDAIAERHGKTLRIHMALNSSGMSRNGVEMTTWSGRGEALQITDQKHLQLVALMTHFAVEDKDDVRKGLAAFNEQTDWLIKHAKLDRSKLTLHAANSFATLEVPEAHLDMVRTGGALFGDTVPTRTEYQRVMQFKSHVAAVHSYPAGNTVGYDRTFTLARDSRLANITVGYSDGYRRVFTNKGHVLINGHRVPVVGKVSMNTLMVDVTDFPDVKGGNEVVLFGKQAGREITQAEIEEINGALLADLYTVWGSSNPKILVD

SEQ ID NO: 13 - DNA, 1227 bp, Pseudomonas putida KT2440(ATCC47954)
atgccctttcgccgtacccttctggctgcatccctggcacttctgatcaccggacaggcccccctgtatgcggcaccaccgttgtcgatggacaacggcaccaacaccctgaccgtgcaaaacagcaatgcctgggtcgaagtcagcgccagcgccctgcagcacaacatccgcacgctgcaggccgagctggccggcaagtccaagctgtgcgccgtgctcaaggccgatgcctatggccacggtatcggcctggtaatgccatcgatcatcgcccaaggcgtgccctgcgtggcggtggccagcaacgaggaggcccgcgtggtccgcgccagtggcttcaccgggcaactggtgcgggtacgcctggccagcctcagcgagctggaagatggcttgcagtacgacatggaagagctggtgggcagcgcggaatttgcccgccaggccgatgccatcgccgcgcgccatggcaagaccttgcgcattcacatggcgctcaactccagcggcatgagccgcaacggggtggagatggccacctggtccggccgtggcgaagcgctgcagatcaccgaccagaagcacctcaagctggtcgcgctgatgacccacttcgccgtggaagacaaggacgatgtacgcaagggcctggcggcattcaacgagcagaccgactggttgatcaagcacgccaggctggaccgcagcaagctcaccctgcacgccgccaactcgttcgctacgctggaagtgccggaagcgcgcctggacatggtacgaacgggtggcgcgctgttcggcgacaccgtgccggcgcgcaccgagtacaaacgtgcgatgcagttcaaatcgcacgtggcggcggtgcacagctatccggccggcaacaccgtgggctatgaccgcaccttcaccctggcccgtgattcgcggctggccaacattacggtcgggtactccgatggctaccgccgggtattcaccaacaagggccatgtgctgatcaacggccaccgtgtgccggtcgtgggcaaggtgtcgatgaacacgctgatggtcgatgtcaccgacttccctgatgtgaaggggggtaacgaagtggtgctgttcggcaagcaggccgggggcgaaatcacccaggccgagatggaagaaatcaacggcgcgttgctcgccgatttgtacaccgtatggggcaattccaacccgaagatactcgtcgac

SEQ ID NO: 14 - DNA, 1227 bp, Pseudomonas putida IFO12996
atgcaatttagccgtaccctcctggctgcatccctcgctctgctgatcactggccaggccccgctgtacgccgcaccgcccctgtcgatggacaacggcaccaccgccctgaccgcgcagaacagcaacgcctgggtcgaaatcagtgccggcgcactgcaacacaacatccgtaccttgcaggccgagttgggcggcaagtccaagctgtgcgccgtgctcaaggccgacgcctatggccacggtatcggcctggtgatgccgtcgatcatcgcccagggcgtgccctgcgtggcggtggccagcaacgaggaggcacgcgtggtccgcgccagtggcttcaccgggcaactggtgcgggtacgcctggccagcctcggcgaagtggaagatgccttgcagtacgacatggaagagctggttggcagcgccgagttcgcccgccagctcgatgccatcgccgaacgccacggcaagaccctgcgcattcacatggcgctcaattccagcggcatgagccgcaacggcgtggaaatgaccacctggtccggccggggtgaagcgctgcagatcactgaccagaagcacctccagctggtcgcgctgatgactcacttcgccgtggaagacaaggacgatgtgcgcaaaggcctggcagcgttcaacgaacagaccgactggctgatcaagcacgcgaagcttgatcgcagcaagctcaccctgcatgccgccaactccttcgctacgctggaagtgccggaagcgcacctggacatggtgcgtaccggtggcgcgctgttcggcgacaccgtgccgacgcgcaccgaataccaacgtgtcatgcagttcaagtcgcacgtggcggcggtgcacagctacccggcaggcaacaccgtcggctacgaccgcaccttcaccctggcgcgtgattcgcgcctggccaacatcaccgtgggttactccgatggctaccgccgggtgttcaccaacaagggccatgtgctgatcaacggccaccgagtgccagtggtgggcaaggtgtcgatgaacaccttgatggtcgatgtcaccgatttccccgatgtgaaggggggcaacgaagtggtgctgttcggcaaacaggccgggagggagatcacccaggccgagatagaagaaatcaacggcgcgctgctcgccgacctctacaccgtatggggcagttccaacccgaagattctcgtcgac

SEQ ID NO: 15 - DNA, 26 bp, Artificial Sequence
  Description of Artificial Sequence Synthetic DNA
gcccatatgactgataaaatcgcggt

SEQ ID NO: 16 - DNA, 25 bp, Artificial Sequence
  Description of Artificial Sequence Synthetic DNA
gccaagcttttagtccgccagttcc

SEQ ID NO: 17 - DNA, 33 bp, Artificial Sequence
  Description of Artificial Sequence Synthetic DNA
ggtattgagggtcgcatgactgataaaatcgcg

SEQ ID NO: 18 - DNA, 30 bp, Artificial Sequence
  Description of Artificial Sequence Synthetic DNA
agaggagagttagagccttagtccgccagt

SEQ ID NO: 19 - DNA, 33 bp, Artificial Sequence
  Description of Artificial Sequence Synthetic DNA
ggtattgagggtcgcatggaaaaactgcggggt

SEQ ID NO: 20 - DNA, 30 bp, Artificial Sequence
  Description of Artificial Sequence Synthetic DNA
agaggagagttagagccttacattgtggtt

SEQ ID NO: 21 - DNA, 32 bp, Artificial Sequence
  Description of Artificial Sequence Synthetic DNA
ggtattgagggtcgcatgaagaaaaaagttgg

SEQ ID NO: 22 - DNA, 30 bp, Artificial Sequence
  Description of Artificial Sequence Synthetic DNA
agaggagagttagagccctatccatcactt

SEQ ID NO: 23 - DNA, 28 bp, Artificial Sequence
  Description of Artificial Sequence Synthetic DNA
ggtattgagggtcgcatgcgtgtggggc

SEQ ID NO: 24 - DNA, 33 bp, Artificial Sequence
  Description of Artificial Sequence Synthetic DNA
agaggagagttagagccttattggtgaccggga

SEQ ID NO: 25 - DNA, 25 bp, Artificial Sequence
  Description of Artificial Sequence Synthetic DNA
ccgggatccatgcaatttagccgta

SEQ ID NO: 26 - DNA, 30 bp, Artificial Sequence
  Description of Artificial Sequence Synthetic DNA
ccggaattctcagtcgacgagaatcttcgg

SEQ ID NO: 27 - DNA, 28 bp, Artificial Sequence
  Description of Artificial Sequence Synthetic DNA
ggaacatgttgtttagccgtaccctcct

SEQ ID NO: 28 - DNA, 20 bp, Artificial Sequence
  Description of Artificial Sequence Synthetic DNA
ggcgaattcgtcgacgagat

SEQ ID NO: 29 - DNA, 30 bp, Artificial Sequence
  Description of Artificial Sequence Synthetic DNA
ggtattgagggtcgcatgagagtggctctc

SEQ ID NO: 30 - DNA, 34 bp, Artificial Sequence
  Description of Artificial Sequence Synthetic DNA
agaggagagttagagcctcatactctcacccctt